(19) United States
(12) Patent Application Publication
(10) Pub. No.: US 2006/0275214 A1
(43) Pub. Date: Dec. 7, 2006

(54) Methods of screening for anti-inflammatory drugs and use thereof

(75) Inventors: Paul Gregor, Rehovot (IL); Nicholas Harris, Rehovot (IL); Juraj Koppel, Kosice (SK)

Correspondence Address: HESLIN ROTHENBERG FARLEY & MESITI PC, 5 COLUMBIA CIRCLE, ALBANY, NY, 12203, US

(73) Assignee: Rimonyx Pharmaceuticals LTD., Ness-Ziona (IL)

(21) Appl. No.: 10/541,099

(22) PCT Filed: Dec. 30, 2003
(86) PCT No.: PCT/IL03/01116

§ 371 Date: Jul. 31, 2006

(51) Int. Cl. A61K 51/00; G01N 33/53; G01N 33/574; A61K 38/16; A61K 31/737; A61K 31/727; A61K 39/395; C07K 16/30

(52) U.S. Cl. 424/1.69; 514/8; 514/54; 514/56; 435/7.1; 435/7.23; 424/178.1; 530/391.1

(57) ABSTRACT

The present invention relates to methods for screening small organic compounds capable of inhibiting the interactions of glycosaminoglycans with effector cell adhesion molecules. Specifically, the present invention provides a method for screening of small organic compounds, the compounds inhibit the interaction of heparin with L-selectin or P-selectin. The compounds identified by the methods of the invention are useful for treating inflammatory disorders, autoimmune diseases and cancer.

### FIELD OF THE INVENTION

The present invention relates to the field of drug screening. More particularly, the present invention relates to methods for screening, identification and optimization of small organic molecules that inhibit cell adhesion mediated by glycosaminoglycans, and use of the compounds for the treatment of inflammation, cancer and autoimmune diseases.

### BACKGROUND OF THE INVENTION

The extracellular matrix (ECM) has an important function in providing structural integrity to tissues and in presenting appropriate environmental cues for cell adhesion, migration, growth, and differentiation. All of these aspects rely on the spatiotemporal expression of adhesive as well as anti-adhesive components in extracellular matrices and on the cell surface. Major constituents of ECM include glycosaminoglycans, fibronectin, laminin, collagen and proteoglycans, which mediate and drive specific cell surface receptor-ligand interactions.

Glycosaminoglycans

Glycosaminoglycans (also referred to herein as “GAG” or “GAGs”) are naturally-occurring carbohydrate-based molecules implicated in the regulation of a variety of cellular processes, including blood coagulation, angiogenesis, tumor growth, and smooth muscle cell proliferation, most likely by interaction with effector molecules. GAGs are linear, non-branched chains of repeating two-sugar (disaccharide) units, which may be up to 150 units in length (See, for example, Jackson et al. (1991) Physiological Reviews 71:481-539 and Kjellen et al. (1991) Ann. Rev. Biochem. 60:443-475).

Glycosaminoglycans can be divided into four main classes on the basis of a repeating disaccharide unit in the backbone. Typically, one sugar is an uronic acid, and the other is either an N-acetylglucosamine or an N-acetylgalactosamine. The main classes of GAGs are: (1) heparan sulfate (D-glucuronic acid/N-acetyl- or N-sulfo-D-glucosamine); (2) chondroitin/dermatan sulfate (D-glucuronic acid or L-iduronic acid/N-acetyl-D-galactosamine); (3) keratan sulfate (D-galactose/N-acetyl-D-glucosamine); and (4) hyaluronic acid (glucuronic acid/N-acetyl-D-glucosamine). All GAGs (with the exception of hyaluronic acid) contain sulfate groups variously esterified to the ring hydroxyl groups of the sugars. These negatively charged groups are believed to affect the biological properties attributed to glycosaminoglycans. The naturally occurring forms of GAGs, particularly heparin, heparan sulfate, chondroitin sulfate and dermatan sulfate are, in fact, complex hetero-oligosaccharides composed of mixtures of differentially sulfated sugar residues.

One of the most thoroughly studied glycosaminoglycans is the widely used anticoagulant heparin. Heparin, a highly sulfated form of heparan sulfate, is found in mast cells. Overall, heparin is less abundant than related sulfated polysaccharides, such as heparan sulfate, dermatan sulfate, and chondroitin sulfate, which are synthesized in nearly all tissues of vertebrates. As a commercial product, heparin is a hetero-oligodisaccharide composition of about 20-60 monomeric units. It has no protein associated with it, its anticoagulant properties being ascribed exclusively to the specific sulfation patterns found on the carbohydrate chains. Heparins are widely used therapeutically to prevent and treat venous thrombosis; they are also known to have a variety of potentially useful biological activities beyond their ability to inhibit blood coagulation including, for example, anti-inflammatory activities (Wang, L. et al., 2002, J. Clin. Invest., 110, 127-136) and the ability to block tumor growth (Borsig, L. et al., 2001, Proc Natl Acad Sci USA 98, 3352-3357). The toxicity of heparin, however, at the levels required to manifest these activities in vivo has limited its clinical use.

Heparan sulfate glycosaminoglycans (also referred to herein as “HS-GAGs”) consist of repeating disaccharide units. Relatively small segments of HS-GAGs contain disaccharide units that are the actual binding sites for ligands (usually 3-10 disaccharides out of 40-160 disaccharides). The specificity of the GAG biosynthetic enzymes imposes restrictions on the disaccharide GAG sequence. HS-GAG chains typically contain regions rich in D-Glucuronic acid (GlcA) and N-acetyl-D-glucosamine (GlcNAc), contiguous variable length sequences containing N-sulfo-D-glucosamine (GlcNS) derivatives, and some sections that contain alternating N-Acetylated and N-Sulfated units of glucosamine. Typical HS-GAG chains contain relatively short segments of modified sequences interspersed among large sections of unmodified units. Interestingly, the relative content of N-Acetylated, N-Sulfated, and N-Acetylated/N-Sulfated domains as well as other properties of the chains appears to be a stable characteristic of the cells from which the HS-GAG was obtained (Esko J D and Selleck S B 2002 Annu. Rev. Biochem. 71, 435-71).

HS-GAG chains are assembled while they are attached to a proteoglycan core protein. Heparan Sulfate Proteoglycans (HS-PGs) are ubiquitous macromolecules associated with the cell surface and the ECM of a wide range of cells of vertebrate and invertebrate tissues (Iozzo R V 1998 Annu. Rev. Biochem. 67, 609-652). The basic HS-PG structure consists of a protein core to which several linear heparan sulfate chains are covalently attached. Three major families of proteoglycan core proteins have been characterized: the membrane-spanning syndecans (four members) (David G 1993 FASEB J, 1023-1030), the glycosylphosphatidylinositol-linked glypicans (six members) (David G, ibid), and the basement membrane PGs perlecan and aggrin (Iozzo R V 1994 Matrix Biol. 14, 203-208). Several other HS-GAG-bearing proteoglycans are known as well (e.g., betaglycan and a CD44 splice variant) (Brown T A et al 1991 J Cell. Biol. 113, 207-221). The syndecans can contain up to five GAG chains whereas glypicans typically contain one to three HS chains. The different core proteins are expressed in a cell-type-specific manner. Studies on the involvement of ECM molecules in cell attachment, growth and differentiation revealed a central role of HS-PGs in embryonic morphogenesis, angiogenesis, metastasis, neurite outgrowth and tissue repair (Pernimon N and Bernfield M 2000 Nature 404, 725-728). The heparan sulfate (HS-GAG) chains, unique in their ability to bind a multitude of proteins, ensure that a wide variety of effector molecules cling to the cell surface (Taipale J and Keski-Oja 1997 FASEB J 11, 51-59). The ability of HS-PGs to interact with ECM macromolecules such as collagen, laminin, and fibronectin, and with different attachment sites on plasma membranes suggests a key role for these proteoglycans in the self-assembly and insolubility of ECM components, as well as in cell adhesion and locomotion.

GAG Effector Cell Adhesion Molecules: Selectins

Inflammation, infection and cell proliferative disorders involve cell-to-cell interactions mediated by GAGs. One discrete family of cell adhesion molecules (CAMs), which effect GAG-mediated cell interaction, is the Selectins. In the context of inflammation, selectins are GAG Effector Cell Adhesion Molecules (EQAMs).

CAMs were originally organized into families on the basis of molecular structure. Of the many adhesion molecules that have been described, three have been collected together in a category known as selectins. E-selectin (formerly known as ELAM.1) is expressed on inflamed endothelial cells in response to inflammatory cytokines. P-selectin (formerly known as PADGEM, GMP-140, or CD61) was originally identified on platelets. L-selectin (formerly known as mLHR, Leu8, TQ-1, gp90, MEL, Lam-1, or Lecam-1) is expressed constitutively on leukocytes. The selectins were grouped together on the basis of structural similarity before their binding specificity was elucidated. At the molecular level, all three selectins exhibit a unique mosaic structure consisting of an N-terminal type-C lectin domain, an epidermal growth factor (EGF)-like domain, and multiple short consensus repeat (SCR) domains homologous to those found in complement regulatory proteins (For general reviews, see Lasky, Annu. Rev. Biochem. 64:113, 1995 and Kansas, Blood 88:3259, 1996). Each selectin is regulated differently and participates in a different manner in the process of inflammation or immunity.

The lectin domains of each selectin are believed to be critical to the adhesive functions of the proteins. The molecules or counter-receptors on the surface of a neighboring cell that are specifically bound by selectins during the process of adhesion have not been fully characterized, although selectins have been shown to bind to oligosaccharide structures, especially sialyl Lewis X (Polley et al. (1991) Proc. Nat. Acad. Sci., USA 88: 6224). There is an increasing appreciation for differences in the ligand binding requirements between the selecting. More recently, binding to sulfated sugars, including GAG structures, has been reported (Lasky et al. (1992) Cell 69: 927, Fiezi, T. et al. (1993) J. Cell Biochem. Supp. 17A:372, and Norgard et al. (1993) FASEB Journal 7: A1262). Selectins appear to be an example of an effector protein in which the binding to the carbohydrate ligand is the primary effector function of the molecule.

Other GAG Effector Cell Adhesion Molecules: Integrins, Fibronectin and Cytokines

Integrin receptors are heterodimeric transmembrane receptors through which cells attach and communicate with extracellular matrices and other cells. (See S. B. Rodan and G. A. Rodan, “Integrin Function In Osteoclasts”, Journal of Endocrinology, Vol. 154, S47-S56 (1997). Diamond M. S. et al. (J Cell Biol, 1995, 130, 1473-1482) demonstrated direct interaction between Mac-1 integrin and HS-GAGs and described its biological relevance for neutrophils. Other integrins are also known to have heparin-biding domains.

The extracellular matrix molecule fibronectin is a glycoprotein whose major functional property is to support cell adhesion. Fibronectin contains at least two characterized high affinity heparin-binding domains. Inhibition of fibronectin-heparan interactions may have a therapeutic use in glomerulosclerosis, a severe complication of many immunologically mediated kidney diseases (including graft-versus-host disease), eventually resulting in loss of renal function (Vliet A. I. Kidney Int 2002 61(2):481-9) and deep venous thrombosis, for instance.

The conventional concept regarding cytokines is that they act in solution as diffusible factors. However, recent progress in cytokine research suggests that many cytokines and growth factors can function in a non-diffusible fashion when immobilized on either the cell surface or extracellular matrix (ECM) by binding to HS-GAGs and HS-PGs.

Inflammation: Therapeutic and Pathological Consequences

Neutrophils, white blood cells, are the primary agents of the inflammatory response. Originating in the bone marrow, neutrophils circulate in the blood where they interact reversibly with the vascular endothelium. In response to inflammatory stimuli, neutrophils adhere tightly to the vascular endothelium, migrate (extravasate) through the vessel wall, and subsequently move along a chemotactic gradient toward the inflammatory stimulus where they respond phagocytically. The interaction of neutrophils with vascular endothelial cells is thus an essential initial step in the acute inflammatory response.

While the inflammatory response of leukocytes is vital for the eradication of invading microorganisms, a substantial and convincing body of evidence indicates that inflammatory cells also cause damage to healthy organs and tissues (Harlan, 1985 Blood 65:513-525). The adhesion of activated neutrophils and monocytes to vascular endothelial cells, with the subsequent release of toxic oxidative metabolites and proteases, has been implicated in the organ damage observed in diseases such as adult respiratory distress syndrome (ARDS; shock lung syndrome), glomerulonephritis, acute and chronic allograft rejection; inflammatory skin diseases; rheumatoid arthritis; asthma, atherosclerosis, systemic lupus erythematosus, connective tissue diseases; vasculitis; and ischemia-reperfusion syndromes (limb replantation, myocardial infarction, crush injury, shock, stroke, and organ transplantation). (Reviewed in Harlan, ibid.) The normal inflammatory response, therefore, can be at once therapeutic and injurious. The deleterious effects of inflammation must be treated by intervening directly with the molecular and cellular processes that cause the inflammation.

Anti-Cell Adhesion Therapy of Inflammation

Anti-cell adhesion therapy has proven to be highly effective in the treatment of a number of inflammatory disorders: Brain edema and death produced by bacterial meningitis (Tuomanen et al., 1989 J. Exp. Med. 170:959); tissue edema associated with delayed-type hypersensitivity reactions (Lindborn et al., 1990 Clin. Immunol. Immunopath. 57:105); airway hyperresponsiveness in allergic asthma (Wegner et al., 1990 Science 247:456); remote lung injury following aspiration (Goldman et al., 1991 FASEB J. 5:A509); late-phase bronchoconstriction following antigen challenge (Gundel et al., 1991 J. Clin. Invest. 88:1407); permeability edema in acute lung inflammation (Mulligan et al., 1991 J. Clin. Invest. 88:1396); the development of autoimmune diabetes can be inhibited (Hutchings et al., 1990 Nature 346, 639). Anti-adhesion therapy can also prolong cardiac allograft survival (Flavin et al., 1991 Transplant, Proc. 23:533), attenuate lung damage and dysfunction secondary to oxygen toxicity (Wegner et al., 1991 Am. Rev. Respir. Dis. 143:A544), attenuate renal allograft rejection (Cosimi et al., 1990 J. Immunol. 144:4604), ameliorate antigen-induced arthritis (Jasin et al., 1990 Arthritis Rheum. 33:S34) and protect against vascular injury and death in endotoxic shock (Thomas et al., 1991 FASEB J. 5:A509).

Such anti-cell adhesion therapy is also efficacious in ischemia and reperfusion injury. Such therapy can be used to reduce permeability edema following ischemia-reperfusion of intestine (Hernandez et al., 1987 Am J. Physiol. 253:H699), myocardial damage following myocardial infraction (Winquist et al., 1990 Circulation 82:III; Ma et al. 1990 Cir. Res. 82:III), vascular and tissue damage following hemorrhagic shock and resuscitation (Mileski et al., 1990 Surgery 108:206), central nervous system damage following ischemia-reperfusion of the spinal cord (Clark et al., 1991 Stroke 22:877), edema and tissue damage following frostbite and rewarming (Mileski et al., 1990 Proc. Am. Burn Assoc. 22:164), and infarct size following ischemia-reperfusion of myocardium (Simpson et al., 1990 Circulation 81:226).

Selectins, which are responsible for the initial attachment of blood borne neutrophils to the vasculature, occupy the most critical position in the inflammatory cascade. As such, selectins are the prime target for an anti-adhesion therapy for inflammation. By neutralizing selectin-mediated cell adhesion, the deleterious consequences of inflammation can be ameliorated, or circumvented.

GAGs have important biological roles, particularly in processes such as cell adhesion and migration, via their interactions with GAG specific ECAMs. A specific example of GAG-mediated cell adhesion is the interaction between selectins and GAGs, specifically HS-GAGs, leading to inflammation and autoimmune disorders.

Modulating the interactions between GAGs and various GAG specific ECAMs has a significant therapeutic value. Indeed, several groups developed small peptides with high affinities for heparin or for heparin-like molecules (i.e., PGs, or other GAGs) (see U.S. Pat. No. 5,534,619 and U.S. Pat. No. 5,919,761 to Wakefield et al.; U.S. Pat. No. 5,877,153 to Harris et al.; and WO0045831) to use in a variety of applications for modulating the activities of native GAGs and PGs.

There is still an unmet need to have a method for identifying non-peptide, small synthetic compounds, which are capable of modulating the interactions between GAGs and ECAMs.

### SUMMARY OF THE INVENTION

The present invention is directed to methods for screening, identification and use of small organic compounds that modulate interactions and signaling events mediated by glycosaminoglycans (GAGs), specifically adhesion events involving GAGs and GAG-specific effector cell adhesion molecules. Given the key role of GAGs in many physiological and pathological conditions, such modulator compounds have a therapeutic use in the treatment and prevention of diseases, specifically diseases related to cell adhesion and cell migration.

According to one aspect, the present invention provides a method of screening for small organic compounds that inhibit the interaction of GAGs with GAG specific ECAMs, the method comprising the steps of:
a. contacting a GAG with an ECAM in the presence of at least one small organic compound;
b. measuring the amount of the GAG bound to the ECAM or the amount of the ECAM bound to the GAG, wherein a significant decrease in GAG-ECAM binding in the presence of the organic compound as compared to GAG-ECAM binding in the absence of said organic compound identifies the organic compound as an inhibitor compound inhibiting GAG-ECAM interaction.

According to another aspect, the present invention provides a method of identifying small organic compounds that inhibit the interaction of GAGs with GAG specific ECAMs, the method comprising the steps of:
a. contacting a GAG with at least one small organic compound;
b. removing of unbound organic compound;
c. adding an ECAM; and
d. measuring the amount of the GAG bound to the ECAM or the amount of the ECAM bound to the GAG, wherein a significant decrease in GAG-ECAM binding for the GAG contacted with the organic compound as compared to GAG-ECAM binding for said GAG not contacted with the compound identifies said compound as inhibitor compound inhibiting GAG-ECAM interaction.

According to one embodiment, the GAG may be immobilized before it is contacted with the ECAM. According to another embodiment, the ECAM may be immobilized before it is contacted with the GAG.

According to yet another embodiment, the GAG or the ECAM may be tagged or labeled before measuring GAG-ECAM binding. Tagging may be performed by the use of a dye, a fluorescent dye, a chemiluminescent agent, or a radioactive agent. Tagging of the ECAM may be performed by an antibody directed to the ECAM or by antibodies specific to a protein fused or bound to the ECAM.

According to another embodiment, the small organic compounds screened by the methods of the present invention interact with GAGs selected from the group consisting of heparan sulfate (HS-GAG), heparin, chondroitin sulfate, dermatan sulfate, keratan sulfate, derivatives and oligosaccharide fragments thereof.

According to one currently preferred embodiment, the GAG is HS-GAG or heparin, a derivative or an oligosaccharide fragment thereof.

According to another embodiment, the small compounds screened by the methods of the present invention interact with proteoglycan-containing GAGs, derivatives, or fragments thereof. Preferably, the proteoglycan-containing GAG is a heparan sulfate proteoglycan (HS-PG).

According to a further embodiment, the small organic compound screened by the methods of the present invention inhibits the interaction of a GAG with a GAG specific ECAM, the ECAM is selected from the group consisting of selecting, integrins, fibronectin, cytokines, derivatives and fragments thereof.

According to one currently preferred embodiment, the small compound screened by the methods of the present invention inhibits the interaction of a GAG with L-selectin or P-selectin. It should be appreciated that the small organic compound inhibits the interaction of the GAG with the carbohydrate binding domain, particularly heparin binding domain, of L-selectin or P-selectin.

According to an additional aspect, the present invention provides a pharmaceutical composition comprising as an active ingredient an inhibitor compound capable of inhibiting the interaction of a GAG with a GAG specific ECAM, the compound is identified by a screening method comprising the steps of:
a. contacting a GAG with an ECAM in the presence of at least one small organic compound;
b. measuring the amount of the GAG bound to the ECAM or the amount of ECAM bound to the GAG, wherein a significant decrease in GAG-ECAM binding in the presence of the organic compound as compared to GAG-ECAM binding in the absence of said compound identifies the compound as an inhibitor compound inhibiting GAG-ECAM interaction,
   further comprising a pharmaceutically acceptable diluent or carrier.

According to another aspect, the present invention provides a pharmaceutical composition comprising as an active ingredient an inhibitor compound capable of inhibiting the interaction of a GAG with a GAG specific ECAM, the compound is identified by a screening method comprising the steps of:
a. contacting a GAG with at least one small organic compound;
b. removing of unbound organic compound;
c. adding an ECAM; and
d. measuring the amount of the GAG bound to the ECAM or the amount of the ECAM bound to the GAG, wherein a significant decrease in GAG-ECAM binding for the GAG contacted with the organic compound as compared to GAG-ECAM binding for said GAG not contacted with the compound identifies said compound as inhibitor compound inhibiting GAG-ECAM interaction.
   further comprising a pharmaceutically acceptable diluent or carrier.

According to one embodiment, the pharmaceutical composition comprises an inhibitor compound that inhibits GAG-ECAM binding by interacting with a GAG selected from the group consisting of heparan sulfate (HS-GAG), heparin, chondroitin sulfate, dermatan sulfate, keratan sulfate, derivatives and oligosaccharide fragments thereof.

According to one currently preferred embodiment, the pharmaceutical composition comprises an inhibitor compound that inhibits GAG-ECAM binding by interacting with HS-GAG or heparin, a derivative or an oligosaccharide fragment thereof.

According to another embodiment, the pharmaceutical composition comprises an inhibitor compound that inhibits the interaction of a GAG with a GAG specific ECAM selected from the group consisting of selecting, integrins, fibronectin, cytokines, derivatives and fragments thereof.

According to one currently preferred embodiment, the pharmaceutical composition comprises an inhibitor compound that inhibits the interaction of a GAG with L-selectin or P-selectin.

According to yet additional aspect, the present invention provides methods for modulating cell adhesion and cell migration in vivo or in vitro mediated by interactions of GAGs with GAG specific ECAMs.

According to one embodiment, the present invention provides a method for inhibiting cell adhesion or migration in vitro comprising the step of exposing the cells to a small organic compound that interacts with at least one GAG in an amount sufficient for preventing the interactions of the GAG with at least one GAG specific ECAM.

According to another embodiment, the present invention provides a method for inhibiting cell adhesion or migration in vivo comprising the step of administering a small organic compound that interacts with at least one GAG in an amount sufficient for preventing the interactions of the GAG with at least one GAG specific ECAM.

According to a further embodiment, cell adhesion or migration is inhibited by the interaction of the small organic compound identified by the methods of the present invention with a GAG selected from the group consisting of heparan sulfate (HS-GAG), heparin, chondroitin sulfate, dermatan sulfate, keratan sulfate, derivatives and oligosaccharide fragments thereof.

According to one currently preferred embodiment, cell adhesion or migration is inhibited by the interaction of the small organic compound identified by the methods of the present invention with HS-GAG or heparin, a derivative or an oligosaccharide fragment thereof.

According to yet another embodiment; cell adhesion or migration is inhibited by the interaction of the small organic compound identified by the methods of the present invention with proteoglycan containing GAG, preferably HS-PG.

According to another embodiment, cell adhesion or migration is inhibited by the small compound identified by the methods of the present invention that inhibit the interaction of a GAG with a GAG specific ECAM, the ECAM is selected from the group consisting of selecting, integrins, fibronectin, and cytokines.

According to one currently preferred embodiment, the small compounds identified by the methods of the present invention inhibit the interaction of a GAG with L-selectin or P-selectin.

According to another aspect, the present invention provides a method for modulating anticoagulant activity of a glycosaminoglycan in a subject comprising the step of administering a therapeutically effective amount of a pharmaceutical composition according to the invention, thereby modulating the anticoagulant activity of the glycosaminoglycan. Preferably, the compounds of the invention modulate the anticoagulant activity of heparin.

According to a further aspect, the present invention provides a method for the treatment or prevention of disorders related to cell adhesion or migration comprising the step of administering to a subject in need thereof a pharmaceutical composition comprising as an active ingredient a therapeutically effective amount of a small organic compound identified by the methods of the present invention, said compound inhibits the interaction of a GAG with a GAG specific ECAM, thereby preventing cell adhesion or cell migration mediated by the GAG.

According to one embodiment, the small organic compound used for the treatment or prevention of a disorder related to cell adhesion or migration is identified by a screening method comprising the steps of:
a. contacting a GAG with an ECAM in the presence of at least one small organic compound;
b. measuring the amount of the GAG bound to the ECAM or the amount of the ECAM bound to the GAG, wherein a significant decrease in GAG-ECAM binding in the presence of the organic compound as compared to GAG-ECAM binding in the absence of said compound identifies the compound as an inhibitor compound inhibiting GAG-ECAM interaction.

According to another aspect, the small organic compound used for the treatment or prevention of a disorder related to cell adhesion or migration is identified by a screening method comprising the steps of:
a. contacting a GAG with at least one small organic compound;
b. removing of unbound organic compound;
c. adding an ECAM; and
d. measuring the amount of the GAG bound to the ECAM or the amount of the ECAM bound to the GAG, wherein a significant decrease in GAG-ECAM binding for the GAG contacted with the organic compound as compared to GAG-ECAM binding for said GAG not contacted with the compound identifies said compound as inhibitor compound inhibiting GAG-ECAM interaction.

According to one embodiment, the disorder related to cell adhesion or migration is selected from inflammatory processes, an autoimmune processes, cancer, atherosclerosis and platelet-mediated pathologies.

According to another embodiment, the small organic compounds of the present invention are administered for treating or preventing an inflammatory disorder, condition or process exemplified by, but not restricted to septic shock, wound associated sepsis, post-ischemic leukocyte-mediated tissue damage (reperfusion injury; such as myocardial or renal ischemia), frost-bite injury or shock, acute leukocyte-mediated lung injury (e.g., adult respiratory distress syndrome), acute pancreatitis, liver cirrhosis, uveitis, asthma, transplantation rejection, graft versus host disease, traumatic shock, stroke, traumatic brain injury, nephritis, acute and chronic inflammation, including atopic dermatitis, psoriasis, and inflammatory bowel disease.

According to another embodiment, the small compounds of the present invention are administered for treating or preventing an autoimmune process exemplified by, but not restricted to, rheumatoid arthritis, multiple sclerosis, systemic lupus erythematosus, Hashimoto's thyroiditis, Grave's disease, Myasthenia gravis, insulin resistance, and autoimmune thrombocytopenic purpura.

According to yet another embodiment, the small organic compounds of the present invention are administered for treating or preventing cancer exemplified by, but not limited to, leukemia, Hogdkin's lymphoma, non-hodgkin's lymphoma, Burkitt's lymphoma, breast cancer, cervical cancer, ovarian cancer, lung cancer, prostate cancer, colon cancer, and uterine cancer.

According to yet another embodiment, the small organic compounds of the present invention are administered for treating or preventing of other diseases which involve cell adhesion processes including, but not limited to, bone degradation, restenosis, eczema, osteoporosis, and osteoarthritis.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 shows L-selectin binding to immobilized heparin.

FIG. 2 demonstrates inhibition of L-selectin binding to immobilized heparin by soluble heparin.

FIG. 3 shows inhibition of L-selectin binding to heparin by anti-L-selectin antibody DREGG-55. Anti-beta-amyloid antibodies were used as a control.

FIG. 4 shows inhibition of P-selectin binding to immobilized heparin by soluble heparin.

FIG. 5 shows an inhibition curve of L-selectin binding to immobilized heparin by an L-selectin inhibitor Compound no. 12.

FIG. 6 shows an inhibition curve of P-selectin binding to immobilized heparin by a P-selectin inhibitor Compound no. 21.

FIG. 7 shows a dose-dependent inhibition effect of Compound no. 5 on neutrophil infiltration in mouse peritonitis.

FIG. 8 demonstrates the anti-inflammatory properties of Compound no. 5 in Delayed Type Hypersensitivity.

FIG. 9 shows the binding of L-selectin to immobilized heparin in the absence or presence of Compound no. 117 (100 μM).

FIG. 10 shows inhibition of L-selectin binding to immobilized bovine kidney heparan sulfate by different inhibitor Compounds.

FIG. 11 demonstrates the therapeutic effect of test Compound no. 11 (TC11) in a mouse model of Inflammatory Bowel Disease (IBD)

### DETAILED DESCRIPTION OF THE INVENTION

The present invention provides methods for screening and identifying compounds capable of inhibiting the interaction between GAGs and GAG specific effector cell adhesion molecules (ECAMs), thereby capable of inhibiting GAG-mediated cell adhesion and cell migration.

Definitions

In accordance with the present invention and as used herein, the following terms are defined with the following meanings, unless explicitly stated otherwise.

The term “compound” refers to small organic molecule having a molecular weight less than 1500 Daltons and preferably between 300 to 1200 Daltons.

The term “GAG” refers to glycosaminoglycans, including heparan sulfate (HS-GAG), heparin, chondroitin sulfate, dermatan sulfate and keratan sulfate. It includes the GAG chains of proteoglycans such as heparan sulfate proteoglycan or chondroitin sulfate proteoglycan. It includes fragments of GAG produced chemically or enzymatically. It also includes derivatives of GAG, which may be produced by chemical or enzymatic means as known in the art.

The term “HS-GAG” refers to heparan sulfate GAG. It includes fragments of heparan sulfate such as those that may be produced chemically, enzymatically or during purification. It includes the HS-GAG chains of proteoglycans such as heparan sulfate proteoglycans. HS-GAG may be free or attached to a linker, support, cell or protein, or otherwise chemically or enzymatically modified. HS-GAGs may be crude or purified from organs, tissues or cells.

“HS-PG” refers to heparan sulfate proteoglycans.

“Heparin” is polysulfated polysaccharide, with no protein associated with it. According to the invention, heparin may be prepared from different organs or species, for example, heparin may be prepared from porcine intestinal mucosa. The invention encompasses heparins with various molecular weights including low molecular weight heparins, such as commercially available Fraxiparin, and other heparin derivatives, prepared or modified by chemical or enzymatic reactions as known in the art.

“GAG Derivatives” consist of products derived from GAGS or ECAMS, respectively, made by one or more chemical or enzymatic modifications. The modifications are designed to modify the activity of relevant groups of the molecules.

“Oligosaccharide fragments” are products made from GAGs by controlled cleavage and preferably purified after cleavage.

The terms “L-selectin/IgG” and “P-selectin/IgG” refer to a selectin chimera molecule, in which an N-terminal portion of the selectin comprising the binding domain is fused to an IgG Fc region (Aruffo et al., Cell 67:35, 1991 and Foxall et al. J. Cell Biol. 117:895, 1992).

The term “GAG specific ECAM” means an effector cell adhesion molecule and refers to a carbohydrate-binding protein molecule involved in mediating cell adhesion, cell-cell and cell-matrix interaction and having a GAG binding domain. Examples of ECAMs are selectins such as L-selectin, P-selectin, integrins, fibronectin, cytokines, and the like. The term “GAG specific ECAM” also includes mutant ECAMs, protein domains, polypeptides or peptides derived from ECAMs, chemical or enzymatic derivatives of ECAM, and the like, so long as the mutant ECAMs, protein domains, polypeptides, peptides and derivatives of ECAMs retain the capability to bind GAGs.

The term “inhibitor Compound” refers to a small organic molecule inhibiting the interaction (binding) between two molecules: (1) a GAG, exemplified by, but not limited to, heparin or HS-GAG; and (2) an ECAM, exemplified by, but not limited to, L-selectin, P-selectin or integrin.

The term “synthetic chemical compound collection” or “compound collection” refers to a collection of random and semi-random synthetic molecules wherein each member of such collection or library is produced by chemical or enzymatic synthesis.

The terms “inflammation”, “inflammatory diseases”, “inflammatory condition” or “inflammatory process” are meant a physiological or pathological condition, which is accompanied by an inflammatory response. Such conditions include, but are not limited to, sepsis, ischemia-reperfusion injury, Crohn's disease, arthritis, multiple sclerosis, cardiomyopathic disease, colitis, infectious meningitis, encephalitis, acute respiratory distress syndrome, the various organ/tissue transplants (such as skin grafts, kidney, heart, lung, liver, bone marrow, cornea, pancreas, small bowel, organ/tissue rejection), an infection, a dermatose, stroke, traumatic brain injury, inflammatory bowel disease and autoimmune diseases.

The term “treatment” or “treating” is intended to include the administration of the compound of the invention to a subject for purposes which may include prophylaxis, amelioration, prevention or cure of disorders mediated by cell adhesion or cell migration events, specifically adhesion events mediated by selectin, more specifically L-selectin or P-selectin-mediated adhesion events. Such treatment need not necessarily completely ameliorate the inflammatory response or other responses related to the specific disorder. Further, such treatment may be used in conjunction with other traditional treatments for reducing the disease or disorder condition known to those of skill in the art.

The methods of the invention may be provided as a “preventive” treatment before detection of, for example, an inflammatory state, so as to prevent the disorder from developing in patients at high risk for the same, such as, for example, transplant patients.

The term “cancer” refers to various cancer-associated conditions including metastasis, tumor growth, and angiogenesis. According to the invention, cancer is exemplified by leukemias, which may be treated successfully with the compounds screened by the methods of the invention.

As used through this specification and the appended claims, the singular forms “a”, “an” and “the” include the plural unless the context clearly dictates otherwise. Thus, for example, reference to “a compound” includes mixtures of such compounds, reference to “a P-selectin”, or “an L-selectin” includes reference to respective mixtures of such molecules, reference to “the formulation” or “the method” includes one or more formulations, methods and/or steps of the type described herein and/or which will become apparent to those persons skilled in the art upon reading this disclosure.

Methods for Compound Screening and Drug Discovery

Currently, attempts for modulating GAG interactions with GAG specific ECAMs are indirect, targeting the heparin-binding domains associated with GAG specific ECAMs by using GAG-mimetics such as heparins, its derivatives and other sulfated GAG mimetics. Another approach (International Patent Application No. WO 02/076173) discloses peptide derivatives that inhibit GAG molecules, specifically hyaluronic acid (HA).

The present invention provides a method for screening and identifying compounds for drug development, disclosing GAGs, specifically HS-GAGs, as novel molecular targets for such screening. The direct targeting of GAGs as described herein is of critical importance since modern drug discovery requires the precise knowledge of the molecular nature of the drug-binding site for efficient drug screening and chemical optimization program.

According to one aspect, the present invention provides a method of screening for small compounds that inhibit the interaction of GAGs with GAG specific ECAMs, the method comprising the steps of:
a. contacting a GAG with an ECAM in the presence of at least one small organic compound;
b. measuring the amount of the GAG bound to the ECAM or the amount of the ECAM bound to the GAG, wherein a significant decrease in GAG-ECAM binding in the presence of the organic compound as compared to GAG-ECAM binding not in the absence of said compound identifies the compound as an inhibitor compound inhibiting GAG-ECAM interaction.

According to another aspect, the present invention provides a method of identifying small organic compounds that inhibit the interaction of GAGs with GAG specific ECAMs, the method comprising the steps of:
a. contacting a GAG with at least one small organic compound;
b. removing of unbound organic compound;
c. adding an ECAM; and
d. measuring the amount of the GAG bound to the ECAM or the amount of the ECAM bound to the GAG, wherein a significant decrease in GAG-ECAM binding for the GAG contacted with the organic compound as compared to GAG-ECAM binding for said GAG not contacted with the compound identifies said compound as an inhibitor compound inhibiting GAG-ECAM interaction.

The screening methods for identification of inhibitor compounds may be performed by various assays, which are well known to one skilled in the art. The assays can be either direct binding assays or inhibition assays. The GAG molecule may be immobilized, or ECAM may be immobilized, or both GAG and ECAM may be present in solution. The detection may focus either on GAG or on ECAM by using various detection methods such as antibodies specific to either GAG or ECAM, biotin-streptavidin, radiolabeling, fluorescent label, and the like. The detection methods may also differ and may be exemplified by spectrophotometry, chemoluminiscence, fluorescence, radioactive detection, and the like. Immobilized GAGs may be used coated on plates or coupled to beads. GAGs may be linked to a carrier such as a protein, using different chemical methods known in the art. Alternatively, the ECAMs may be immobilized, for instance by coating to plates or coupling to beads. ECAMs may be used as fusion proteins or domains containing the GAG-binding domain. Another useful approach may be to use as a source of GAG a whole cell such as an endothelial cell. This is particularly relevant for identifying inhibitor Compounds that prevent adhesion to such endothelial cells. Inhibition of human lymphocyte rolling on endothelial cells under shear flow with inhibitor Compounds is exemplified herein below (Example 16).

According to one embodiment, compounds for screening may be produced by synthetic chemistry or may be natural compounds, individual or in mixtures, pre-selected by an algorithm, compressed libraries and the like. A preferred method of screening is known as High-Throughput Screening (HTS), in which thousands of compounds are screened with the aid of robotics.

According to one currently preferred embodiment, compound screening according to the method of the present invention is used as iterative screening in conjunction with chemical optimization via synthetic chemistry.

According to another embodiment, the small organic molecules screened by the methods of the present invention interact with GAGs selected from the group consisting of heparan sulfate (HS-GAG), heparin, chondroitin sulfate, dermatan sulfate, keratan sulfate, derivatives and oligosaccharide fragments thereof.

According to one currently preferred embodiment, the GAG is HS-GAG or heparin, a derivatives or an oligosaccharide fragment thereof.

The GAGs may be crude or purified from an organ, tissue or cell such as human liver, human brain, endothelial cells and the like. The GAGs may be produced synthetically or commercially available. The present invention also encompasses GAG derivatives or fragments, which may be produced by chemical or enzymatic means known in the art so long as the binding of the GAG derivatives or fragments to ECAMs is preserved.

According to another embodiment the small organic compounds screened by the methods of the present invention interact with a proteoglycan-containing GAG. Preferably, the proteoglycan-containing GAG is heparan sulfate proteoglycan (HS-PG). Proteoglycans containing GAG chains may be obtained from an organ, tissue, cell or tumor in a crude or purified form. Examples for HS-PGs are syndecan or aggrin. Proteoglycans having other GAG chains, such as versican, may be also used.

Many important regulatory proteins bind tightly to heparin including, for example, chemokines, growth factors, cytokines, enzymes, and proteins involved in lipid metabolism. Although interactions of proteins with GAGs such as heparin and heparan sulfate are of great biological importance, the structural requirements for protein-GAG binding have not been well characterized. Ionic interactions are important in promoting protein-GAG binding and the spacing of the charged residues may determine protein-GAG affinity and specificity. Consensus sequences such as XBBBXXBX (X=any amino acid, B=arginine, lysine or histidine) and XBBXBX are found in some protein sites that bind GAG. Another consensus sequence TXXBXXTBXXXTBB (T=turn in the secondary protein structure) is described, where turns bring basic interacting amino acid residues into proximity.

It was first suggested that specific protein binding sequences might exist in the carbohydrate chain of heparin by the observation that some preparations were more effective than others in inhibiting coagulation. Careful studies in 1987 revealed that there is a defined five-sugar sequence (i.e., pentasaccharide) with a characteristic sulfation pattern that represents the specific binding site for AT III, a protease inhibitor that blocks the action of thrombin and other enzymes that initiate blood coagulation. Although weaker and less specific binding of these proteins to other regions of heparin can occur, virtually all of the anticoagulant activity of heparin is attributable to this five-sugar sequence.

Similarly, it was anticipated that unique sequences of extracellular HS-GAGs bind specifically to important proteins, including growth factors, cytokines and many other signaling molecules, and by doing so influence fundamental biological processes (Sasisekharan, R. and Venkataraman, G., Current Opinion in Chem. Biol., 2000, 4, 626-631; Lindahl, U. et al., 1998, J. Biol. Chem., 273, 24979-24982; Esko, J. and Selleck, S. B., 2002, Annu. Rev. Biochem., 71, 435471). The methods of the present invention for screening and identifying compounds capable of inhibiting GAGs, specifically HS-GAG, provide new approaches and strategies for therapeutic intervention at the cell-tissue-organ interface.

According to one embodiment, the small organic molecules screened by the methods of the present invention inhibit the interaction of GAGs with GAG specific ECAMs selected from the group consisting of selecting, integrins fibronectin, and cytokines. According to the invention the GAG specific ECAMs may be the full length proteins, however, they may also be derivatives and/or fragments of ECAMs, which are produced chemically or enzymatically as known in the art so long as the binding to GAGs is preserved. The present invention also encompasses ECAMs produced synthetically or ECAMs produced by recombinant DNA technology expression (see Maniatis, et al., Molecular Cloning; A Laboratory Manual (Cold Spring Harbor Lab, New York, 1982).

According to one currently preferred embodiment, the small compounds screened by the methods of the present invention inhibit the interaction of GAGs with L-selectin or P-selectin, namely the interaction of the GAGs with the carbohydrate binding domain, particularly heparin binding domain, of L-selectin or P-selectin.

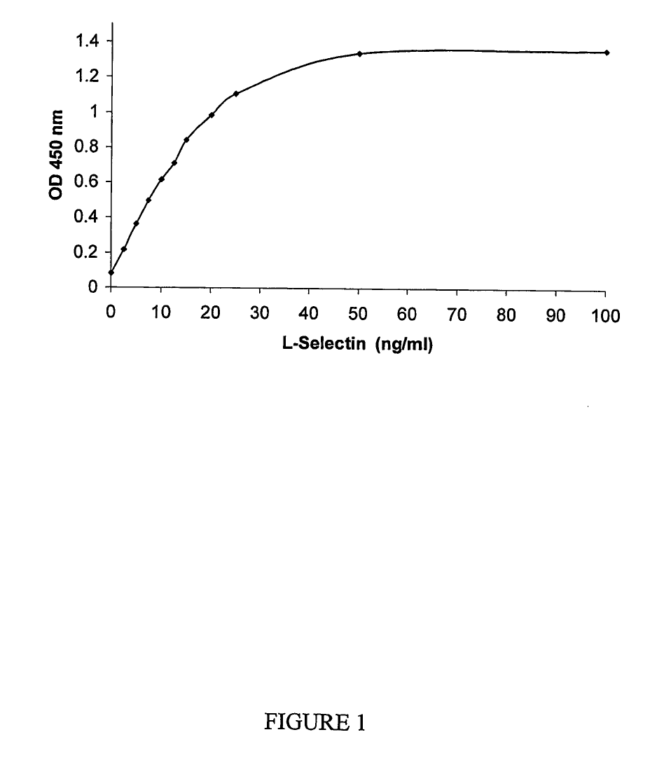

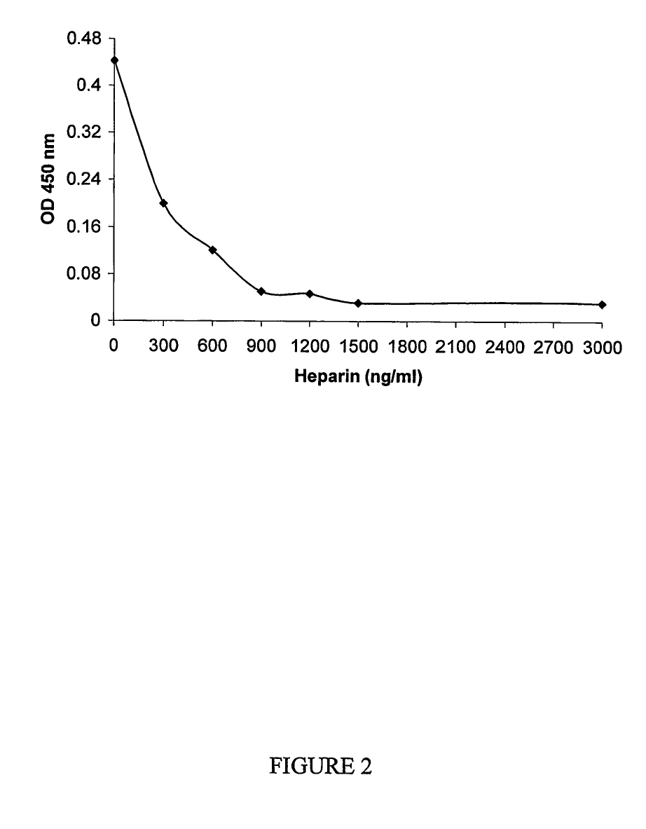

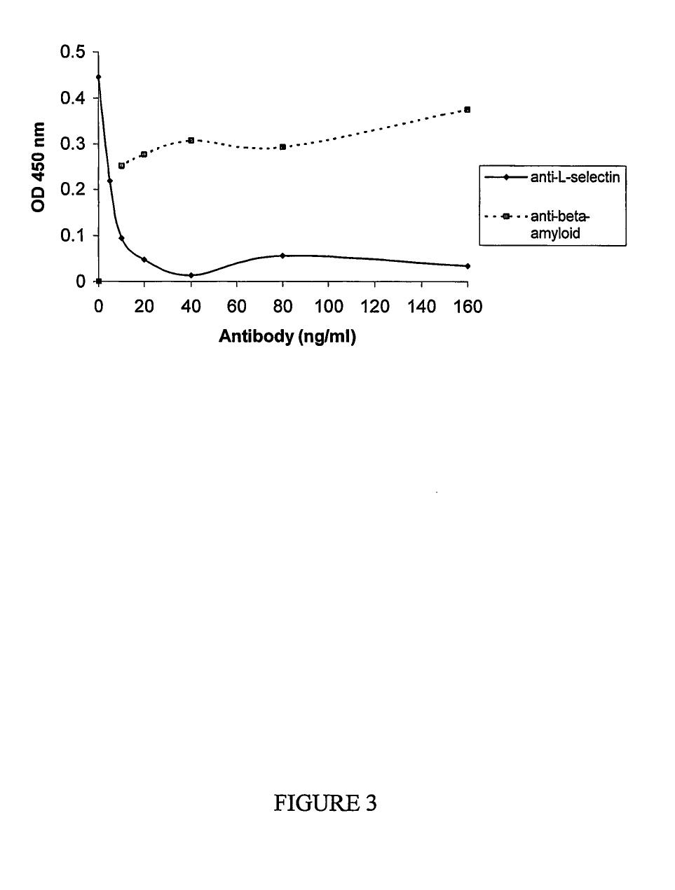

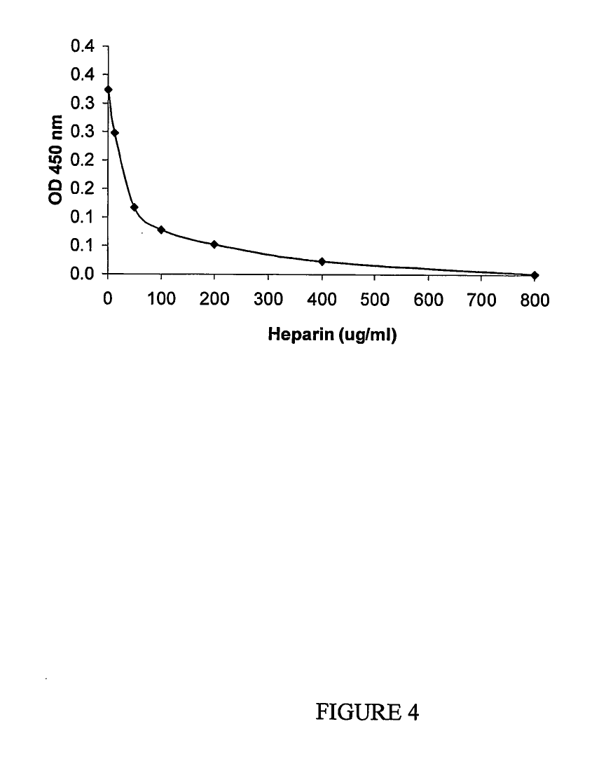

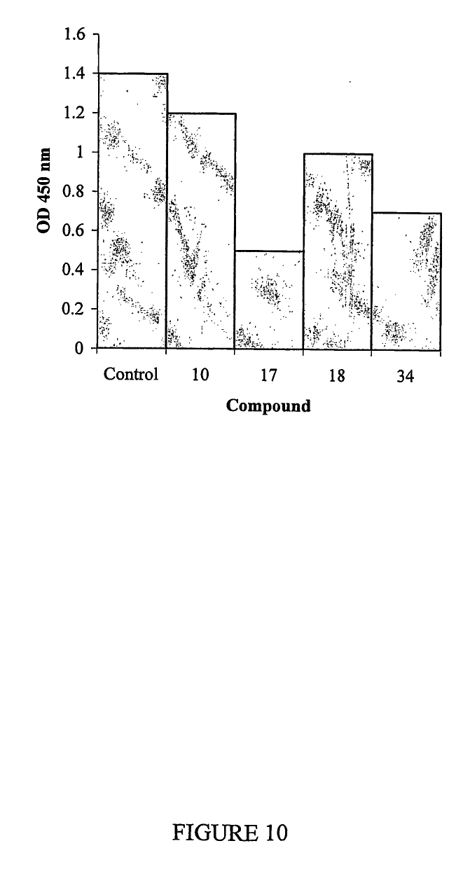

Assays for L-selectin binding to heparin have been previously described (Green P J et al, 1995, Glycobiology 5 (1): 29-38; Weiz-Schmidt G et al, 1999, Anal. Biochem 273(1); 81-88); however, the present invention discloses, for the first time, the use of L-selectin-GAG binding assays for identification of inhibitor Compounds that are targeting GAG binding sites. The screening method of the present invention is based on an ELISA assay for L-selectin interaction with heparin on 96-well plates, the method is suitable for screening compound collections, newly developed by the inventors of the present invention. The assay measures binding of L-selectin to immobilized heparin. The amount of bound L-selectin is determined by an ELISA assay using a monoclonal antibody conjugated to horseradish peroxidase. FIG. 1 shows the saturation curve of L-selectin binding to heparin. Soluble heparin inhibited L-selectin binding to immobilized heparin (FIG. 2). A monoclonal antibody (mAb) directed against the carbohydrate-binding domain of L-selectin (DREGG-55) inhibited L-selectin binding to heparin (FIG. 3), providing a further confirmation of the specificity of binding. This method can be used with other GAG specific ECAMs such as P-selectin, integrins or fibronectin. For instance, an assay for P-selectin binding to heparin has also been developed, and FIG. 4 shows inhibition of P-selectin binding by soluble heparin. Additionally, other GAGs are capable of replacing heparin in this kind of assay (see FIG. 10). In place of heparin one may immobilize a different HS-GAG such as purified HS-GAG from an organ, tissue or cell of interest. HS-GAGs may be immobilized by methods similar for immobilization of heparin as described in the Examples herein below, or by other means known in the art.

Preferably, when using this kind of an assay for compound screening, one may use a particular GAG or PG from a target tissue, such as endothelial cell HS-GAG, kidney purified HS-GAG, HS-PG, and the like. The reason is that molecular diversity of HS-GAGs is regulated in a tissue and cell-specific manner and different HS-GAGs have different binding sites for GAG specific ECAMs.

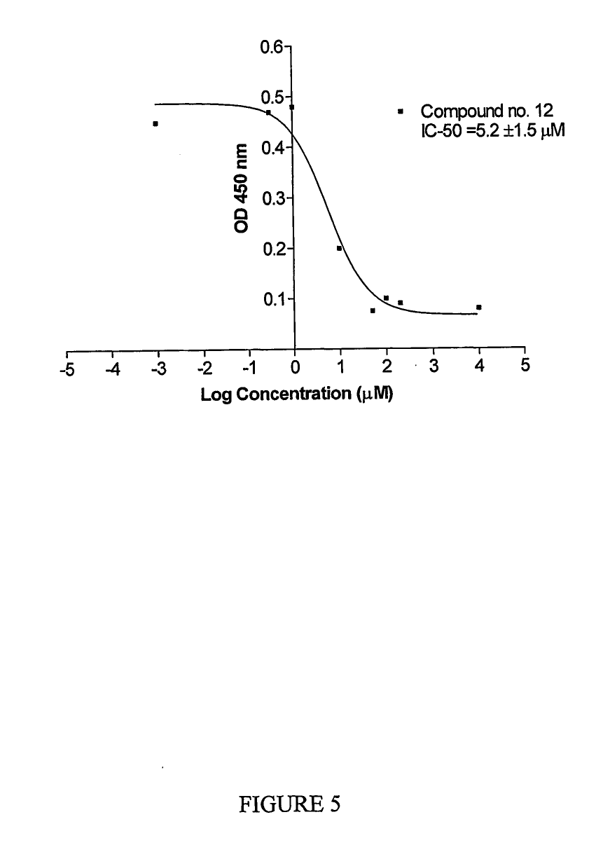

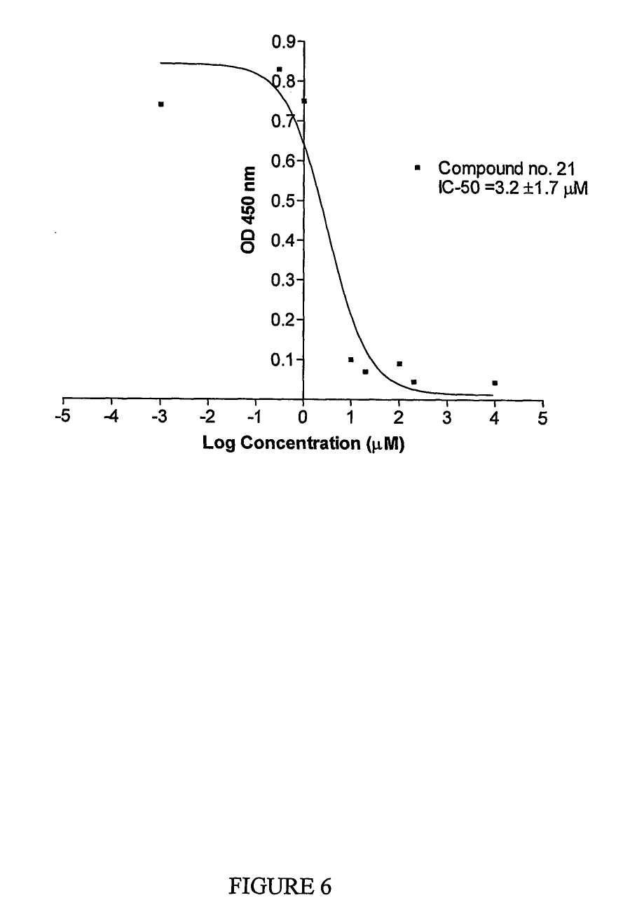

The present invention demonstrates, for the first time, that this kind of GAG-protein interaction assay is suitable for screening collections of compounds and for discovery of novel drugs. As described herein below, the L-selectin assay was used to screen a collection of several thousand compounds on 96-well plates. For this purpose, test compounds were co-incubated with L-selectin/IgG in individual wells containing immobilized heparin. Following completion of the assay and color development, percentage of inhibition obtained for each compound was determined. Positive and negative controls were included on every plate. Compounds, which inhibited at least 30% of the binding as detected by color intensity, were scored as hits and selected for further analysis. Dose-response inhibition curves were generated for these hit compounds as exemplified in FIGS. 5 and 6. Compounds having IC-50 values (i.e., the concentration of a compound which results in 50% inhibition of binding as compared to the binding in the absence of the compound) in the range of 0.5-20 μM were generally suitable for further development and chemical optimization.

According to one embodiment of the present invention, the inhibitor compounds identified by the methods of the present invention interact with GAGs and inhibit their interaction with GAG specific ECAMs.

In principle, the inhibitor compounds can inhibit L-selectin-heparin interaction either (i) by direct binding to heparin and thus preventing its interaction with L-selectin; or (ii) by direct binding to L-selectin and subsequently preventing its interaction with heparin (a third theoretical possibility is that the compound binds to both heparin and L-selectin, but this is statistically a very rare possibility).

Compounds found to be suitable for further development and chemical optimization may be further subjected to a second screening, identifying compounds that directly bind to heparin. This was performed as follows: individual test compounds were incubated with immobilized heparin in the absence of L-selectin/IgG. Following washing of the plates to remove all unbound compound, L-selectin/IgG was added and the standard assay protocol was followed. As exemplified herein below, compounds which were found to inhibit heparin-L-selectin binding in the co-incubation assay were found to have the same binding capabilities under the pre-incubation conditions, as manifested by their very similar IC-50's values under pre-incubation or co-incubation conditions. These results show that the compounds inhibit the L-selectin-heparin interaction by direct binding to heparin and not by binding to L-selectin/IgG. Furthermore, the interactions of the compounds with heparin were resistant to washing and therefore relatively tight.

According to yet further aspect the present invention provides a pharmaceutical composition comprising as an active ingredient an inhibitor compound identified by a screening method comprising the steps of:
a. contacting a GAG with an ECAM in the presence of at least one organic compound;
b. measuring the amount of the GAG bound to the ECAM or the amount of the ECAM bound to the GAG, wherein a significant decrease in GAG-ECAM binding in the presence of the organic compound as compared to GAG-ECAM binding in the absence of said compound identifies the compound as an inhibitor compound inhibiting GAG-ECAM interaction,
   further comprising a pharmaceutically acceptable diluent or carrier.

The present invention also provides a pharmaceutical composition comprising as an active ingredient an inhibitor compound identified by a screening method comprising the steps of contacting a GAG with at least one small organic compound, removing of the unbound organic compound, adding an ECAM, and finally measuring the binding of GAG-ECAM for the GAG contacted with the compound, wherein if said binding is lower than the GAG-ECAM binding for the same GAG, which has not been contacted with the compound, than said compound is an inhibitor compound, the composition further comprising a pharmaceutically acceptable diluent or carrier.

The term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic agent is administered. Such pharmaceutical carriers can be sterile liquids, such as water and oils, including those of animal, vegetable or synthetic origin, such as peanut oil, soybean oil, mineral oil, sesame oil, petroleum, and the like. Water is a preferred carrier when the pharmaceutical composition is administered intravenously. Saline solutions and aqueous dextrose and glycerol solutions can also be employed as liquid carriers, particularly for injectable solutions.

Suitable pharmaceutical excipients include, but are not limited to, starch, glucose, lactose, sucrose, gelatin, silica gel, sodium stearate, glycerol monostearate, talc, sodium chloride, glycerol, propylene, glycol, water, ethanol, and the like. The composition, if desired, can also contain minor amounts of wetting or emulsifying agents, or pH buffering agents.

The compositions can take the form of solutions, suspensions, emulsion, tablets, pills, capsules, powders, sustained-release formulations, and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, and the like. Examples of suitable pharmaceutical carriers are described in “Remington's Pharmaceutical Sciences” by E. W. Martin. Such compositions will contain a therapeutically effective amount of the compound, preferably in a purified form, together with a suitable amount of a carrier so as to provide the form for proper administration to the subject. The formulation should suit the mode of administration.

According to one embodiment, the pharmaceutical composition comprises an inhibitor compound that inhibits GAG-ECAM binding by interacting with GAGs selected from the group consisting of heparan sulfate (HS-GAG), heparin, chondroitin sulfate, dermatan sulfate, keratan sulfate, derivatives and oligosaccharide fragments thereof.

According to one currently preferred embodiment, the pharmaceutical composition comprises an inhibitor compound that inhibits GAG-ECAM binding by interacting with HS-GAG or heparin, a derivative or an oligosaccharide fragment thereof.

According to another embodiment, the pharmaceutical composition comprises an inhibitor compound that inhibits the interaction of GAGs with GAG specific ECAMs selected from the group consisting of selectins, integrins, fibronectin, and cytokines.

According to one currently preferred embodiment, the pharmaceutical composition comprises an inhibitor compound that inhibits the interaction of GAGs with L-selectin or P-selectin.

As exemplified for the first time by the present invention, structurally diverse compounds are capable of inhibiting GAG interactions with ECAMs. Since GAGs and ECAMs have been implicated in a multitude of disorders, the inhibitor compounds of the invention have therapeutic implications and are useful for treating a variety of disorders.

Methods for Modulating Cell Adhesion and Cell Migration

According to another aspect, the present invention provides methods for modulating cell adhesion or cell migration in vitro or in vivo mediated by interactions of GAGs with GAG specific ECAMs.

According to one aspect, the present invention provides a method for inhibiting cell adhesion or migration in vitro comprising the step of exposing the cells to a small organic compound that interacts with at least one GAG in an amount sufficient for preventing the interactions of the GAG with at least one GAG specific ECAM. Examples of such inhibitor Compounds are given herein below in Example 16.

According to another embodiment, the present invention provides a method for inhibiting cell adhesion or migration in vivo comprising the step of administering a small organic compound that interacts with at least one GAG in an amount sufficient for preventing the interactions of the GAG with at least one GAG specific ECAM. Examples of such inhibitor Compounds are given herein below in Examples 9 and 10.

According to one embodiment, cell adhesion or migration is inhibited by the interaction of the small compounds identified by the methods of the present invention with GAGs selected from the group consisting of heparan sulfate (HS-GAG), heparin, chondroitin sulfate, dermatan sulfate, keratan sulfate, derivatives and oligosaccharide fragments thereof.

According to one currently preferred embodiment, cell adhesion or migration is inhibited by the interaction of the small organic compounds identified by the methods of the present invention with HS-GAG or heparin, a derivatives or an oligosaccharide fragment thereof.

According to yet another embodiment, cell adhesion or migration is inhibited by the interaction of the small organic compounds identified by the methods of the present invention with proteoglycan containing GAG, preferably HS-PG.

According to one embodiment, cell adhesion or migration is inhibited by compounds identified by the methods of the present invention that inhibit the interaction of GAGs with GAG specific ECAMs selected from the group consisting of selecting, integrins, fibronectin, and cytokines.

According to one currently preferred embodiment, the small compounds identified by the methods of the present invention inhibit the interaction of GAGs with L-selectin or P-selectin.

Emerging evidence indicates that GAGs, and in particular HS-GAG, are carbohydrate receptors with which the selectins interact (Nelson R M, et al., 1993, Blood 82, 3253-3258; Ma, Y Q and Geng, J G, 2000, J. Immunol. 165, 558-565; Kawashima H., et al., 2000, J. Biol. Chem., Aug 18 issue; Giuffre, L. et al., 1997, J. Cell. Biol. 136, 945-956; Watanabe N., et al., 1999, J. Biochem. 125, 826-831; Li Y F et al., 1999, FEBS Lett 444, 201-205). Consistent with this observation, heparin, HS-GAG, and heparin-derived oligosaccharides block L-selectin-dependent adhesion directly (Bevilacqua et al, 1996, U.S. Pat. No. 5,527,785). Furthermore, short sulfated heparin-derived tetrasaccharides reduced binding of neutrophils to COS cells expressing P-selectin (Nelson R M, et al., 1993, Blood 82, 3253-3258). The multivalent nature of HS-GAG may be an important factor in binding L-selectin under flow conditions (Sanders et al, ibid). The endothelial proteoglycans recognized by L-selectin are HS-PGs, rather than sialylated, fucosylated or sulfated glycoprotein ligands (Koenig, A., et al., 1998, J. Clin. Invest. 101, 877-889). Endothelial HS chains bind L-selectin and P-selectin but not E-selectin (Koenig et al., ibid). In vivo administration of heparinase III exerts endothelial and cardioprotective effects in feline myocardial ischemia-reperfusion injury. This type of injury is known to be mediated via L- and P-selectins and the heparinase effect is probably due to degradation of HS (Hayward, R. et al., 1997, J. Pharmacol Exp. Ther. 283, 1032-1038).

L-selectin has a number of features that are different from the other known selectins. First, the tissue distribution pattern is opposite to that of P- and E-selectin, namely L-selectin is expressed on the surface of leukocytes, rather than on the endothelium, where its ligand is expressed. Second, L-selectin is constitutively expressed, rather than being up regulated during inflammation, and is in fact shed following activation. This may act to allow the activated cells to be released after binding, or may indicate a role of L-selectin in cellular activation. Third, L-selectin is present not only on neutrophils and monocytes, but also on most lymphocytes; while the ligand counterpart is present not only on endothelium but also on lymph node high endothelial venules (HEV). L-selectin appears to play a key role in homing to lymph nodes (Shimizu et al., Immunol. Today 13:106, 1992; Picker et al., Annu. Rev. Immunol. 10:561, 1992). In pathological conditions involving the immune system, it may be L-selectin that plays the most central role.

Methods for Modulating Heparin and GAG Function

The compounds of the present invention may be useful for counteracting the actions of heparin and other anticoagulant glycosaminoglycans on thrombin and Factor Xa activity, and may affect other proteins as well. Heparin is used routinely for anticoagulation. The interactions of exogenously administered heparin with the proteins of the coagulation and fibrinolytic pathways have been summarized in detail (van Kuppevelt, T. H., et al., J Biol Chem, 273 (21): 12960-12966, 1998). It is often necessary to reverse the effects of heparin when anticoagulation has reached a stage at which hemorrhage becomes a threat, notably after the routine use of heparin for anticoagulation during cardiopulmonary bypass, and in patients who develop an endogenous heparin-like coagulation inhibitor. Currently, the only FDA-approved heparin antidote available is Protamine. Protamine is a mixture of basic proteins from fish sperm nuclei that contains a high concentration of the amino acid arginine. When injected into a person who has been treated with heparin, Protamine complexes rapidly to the heparin, thereby neutralizing its activity. Although Protamine is effective in humans against unfractionated heparin, it is not effective against low molecular weight heparins or against the non-heparin glycosaminoglycan anticoagulant Orgaran®, i.e., a mixture of chondroitin sulfate/heparan sulfate/dermatan sulfate. Protamine also has numerous side effects including pulmonary hypotension that are difficult to control and provide significant health risks to the patient. Also, since Protamine is obtained from a natural source, it is a poorly defined and potentially variable product, dosage determination can be problematic. Well-defined heparin- or other GAG-binding compounds could be of considerable utility for reversing overdose of these specific anticoagulant preparations. Carson and co-workers (Munro, M. S., et al., Trans Am Soc Artif Intern Organs, 27: 499-503, 1983) have identified a heparin-binding peptide from an epithelial/endothelial cell surface protein that has some ability to neutralize heparin effects on thrombin generation, but optimal effects were found only at high peptide concentrations and low heparin and low thrombin concentrations. The small organic compounds of the invention would be substantially preferable over these peptides as they are more stable and cost effective. The compounds of the invention can thus be useful in neutralization of unfractionated heparin, low molecular weight heparin, or Orgaran.

Multiple interactions between the proteins of the coagulation and fibrinolysis pathways and endothelial cell surface PGs are normally balanced on the surface of the endothelial cells in order to create a non-thrombotic state. Heparin-binding compounds of the invention could behave similarly to platelet factor 4 (PF4) in that they could bind to heparin, reduce the anticoagulant activity, which occurs on the surface of endothelial cells, and thereby enable a clot to form.

Additional possible use for the compounds of the present invention is to block the uptake and clearance of heparin by blocking heparin receptors in tissues without binding to circulating heparin, and thus to prolong the half-life of heparin in the circulation. Use of the compounds of the invention would reduce the frequency of administration of heparin, as well as the amount needed. This could be especially useful for home-based therapy with low molecular weight heparin, which is administered by subcutaneous injection and is becoming the standard post-hospitalization anticoagulation treatment.

Methods for Treatment of Disorders Related to Cell Adhesion or Cell Migration

According to yet another aspect the present invention provides a method for the treatment or prevention of disorders related to cell adhesion or migration comprising the step of administering to a subject in need thereof a pharmaceutical composition comprising as an active ingredient a therapeutically effective amount of a small organic compound identified by the methods of the present invention that inhibits the interaction of GAGs with GAG specific ECAMs, thereby preventing cell adhesion or cell migration mediated by the GAG.

Anti cell adhesion and anti-cell migration therapy has been proven to be highly effective in the treatment of number of diseases and disorders including inflammatory processes, autoimmune processes, cancer and platelet-mediated pathologies.

According to one embodiment, the small organic compound for the treatment or prevention of a disorder related to cell adhesion or migration is identified by the screening method comprising the steps of:
a. contacting a GAG with an ECAM in the presence of at least one small organic compound;
b. measuring the amount of the GAG bound to the ECAM or the amount of the ECAM bound to the GAG, wherein a significant decrease in GAG-ECAM binding in the presence of the organic molecule as compared to GAG-ECAM binding in the absence of said compound identifies the compound as an inhibitor compound inhibiting GAG-ECAM interaction.

According to another embodiment, the small organic compound for the treatment or prevention of a disorder related to cell adhesion or migration is identified by a screening method comprising the steps of contacting a GAG with at least one small organic compound, removing of the unbound organic compound, adding an ECAM, and finally measuring the binding of GAG-ECAM for the GAG contacted with the compound, wherein if said binding is lower than the GAG-ECAM binding for the same GAG, which has not been contacted with the compound, than said compound is an inhibitor compound.

According to another embodiment, the GAG of the inhibited GAG-ECAM interactions is selected from the group consisting of heparan sulfate (HS-GAG), heparin, chondroitin sulfate, dermatan sulfate, keratan sulfate, derivatives and oligosaccharide fragments thereof.

According to one currently preferred embodiment, the GAG of the inhibited GAG-ECAM interactions is HS-GAG or heparin, a derivative or a fragment thereof.

According to yet another embodiment, the ECAMs of the inhibited GAG-ECAM interactions is selected from the group consisting of selecting, integrins fibronectin, and cytokines.

According to one currently preferred embodiment, the ECAM of the inhibited GAG-ECAM interactions is L-selectin or P-selectin.

Selectins serve as good targets for anti-inflammatory therapeutics. An inflammatory stimuli cause circulating neutrophils to respond by adhering to the vascular endothelium. The adhesion proteins, the selecting, regulate neutrophil and lymphocyte adhesion to and entry into lymphoid tissues and sites of inflammation (Rosen, 1990 Am. J. Respir. Cell. Mol. Biol., 3:397-402). The selectins capture leukocytes in the flowing blood stream and mediate their intermittent attachment to specific sites with consequent leukocyte “rolling” along the endothelial cell surface. A cascade of secondary, tighter cell-adhesive events then follows. P-selectin is a cytoplasmic glycoprotein in endothelial cells and platelets, which can be rapidly translocated to the cell surface upon activation with thrombin (Larsen et al., 1989 Cell 3:397-402; Geng et al., 1990 Nature, 343:757-760). Both P-selectin and E-selectin are adhesion proteins for neutrophils and monocytes (Johnston et al., 1989 Cell 56:1033-1044). A subpopulation of memory T-cells has also been shown to bind E-selectin (Picker et al., 1991 Nature (London) 349:796-799). Data suggest that E-selectin is involved primarily in the acute inflammatory response. E-selectin expression is also rapidly inducible in vivo and coincides with the influx of neutrophils (Cotran et al., 1986, J. Exp. Med. 164:661).

In contrast to vascular selecting, L-selectin is constitutively expressed by leukocytes and mediates lymphocyte adhesion to peripheral lymph node high endothelial venules (Spertini et al., 1991 J. Immunol. 147:2565-2573). L-selectin is constitutively expressed on resting neutrophils in an apparently functional form. Recently, Buerke et al. have demonstrated the important role of selectins in inflammatory states such as ischemia-reperfusion injury in cats (Buerke, M. et al., J. Clin. Invest. (1994) 93:1140). Turunen et al. have demonstrated the role of sLex and L-selectin in site-specific lymphocyte extravasation in renal transplants during acute rejection (Turunen, J. P. et al., Eur. J. Immunol. (1994) 24:1130). P-selectin has been shown to be centrally involved particularly with regard to acute lung injury. Mulligan et al. have reported strong protective effects using anti-P-selectin antibody in a rodent lung injury model. (Mulligan, M. S. et al., J. Clin. Invest., (1991) 90:1600, Mulligan, M. S. et al., Nature (1993) 364:149). A central role of P-selectin in inflammation and thrombosis has been demonstrated by Palabrica et al. (Palabrica, T. et al., Nature (1992) 359:843). Recent publications on selectin ligands describe the use of L-selectin as an indicator of neutrophil activation (U.S. Pat. No. 5,316,913 to Butcher et al.), and assays for the inhibition of leukocyte adhesion (U.S. Pat. No. 5,318,890 to Rosen et al.). The presence of L-selectin and E- or P-selectin ligands on mononuclear cells has implicated these receptor-ligand interactions in chronic inflammation. This has been supported by the finding of chronic expression of E-selectin in dermatological conditions, and P-selectin expression on joint synovial endothelium derived from rheumatoid arthritis patients (L. Lasky Annu. Rev. Biochem. 64:113-39 (1995); M. Forrest and J. C. Paulson in Physiology and Pathophysiology of Leukocyte Adhesion Ed. by D. Niel Grangier and Deert Schmid-Schonbein, Oxford University Press, New York, N.Y. (1995)).

Selectin-mediated leukocyte rolling is the first event in the inflammatory cascade. Given the primacy of selectins in the inflammatory response, this family of adhesion molecules has been earmarked as a target for anti-inflammatory therapy.

Monoclonal antibodies to L-selectin prevent neutrophil emigration into inflamed skin (Lewinsohn et al., 1987 J. Immunol. 138:4313), neutrophil and monocyte emigration into inflamed ascites (Jutila et al., 1989 J. Immunol. 143:3318), and neutrophil emigration into inflamed peritoneum. Monoclonal antibodies to E-selectin inhibit neutrophil migration to the lung and thus provide a basis for their use in prevention or treatment of asthma (Gundel et al., 1991 J. Clin. Invest; Mulligan et al., 1991 J. Clin. Invest. 88:1396). Jasin et al. provide support for the use of antibodies in inhibiting neutrophil accumulation in inflamed synovium (Jasin et al., 1990 Arthritis Rheum. 33:S34; Koch et al., 1991 Lab. Invest. 64:313). Monoclonal antibody EL-246, directed against both L-selectin and E-selectin, attenuated sepsis-induced lung injury (Ridings, P C et al., 1995, Arch Surg. 1199-1208). Monoclonal antibody SMART is an L-selectin blocking antibody in clinical trials for trauma associated with multiple organ failure (this condition is believed to be due in part to infiltration of inflammatory cells). The anti-L-selectin antibody is expected to provide its therapeutic effect by preventing neutrophil adhesion to endothelium and it is active in vivo in a primate model of severe trauma (Critical Care Medicine 1999, 27, 1900-1907). It is believed that this monoclonal antibody will be also useful in the treatment of adult respiratory distress syndrome and myocardial infarction.

Crude fractions of heparin have been reported to bind and inhibit P-selectin-dependent and L-selectin-dependent interactions. The specificity of heparin binding to the selectins is unclear; although other sulfated polysaccharides such as fucoidan and dextran sulfate also bind to P-selectin and L-selectin. Norgard-Sumnichi K E and Varki A. (JBC 1995, 270, 12012-12024); Norgard-Sumnichi K E and Varki A (Science 261, 480-483, 1993); and Koenig A. et al. (1998 J. Clin. Invest.101, 877-889) described interactions of heparin, HS-GAG and oligosaccharide fragments thereof with selectins by affinity chromatography. They described that in affinity chromatography, endothelial heparan sulfate proteoglycans and heparan sulfate fragments bind to L-selectin/IgG immobilized on protein A-Sepharose. Nelson R M et al. (Blood 82, 1993, 3253-3258) described that heparin inhibited the binding of L- and P-selectin to immobilized sLex-BSA. It was concluded that heparin and heparin-derived oligosaccharides bind to L- and P-selectins.

Bevilacqua et al. (U.S. Pat. No. 5,527,785) provide a method of modulating selectin binding in a subject comprising administering heparin-like oligosaccharides. Preferably, the oligosaccharide binds to L- or P-selectin.

Xie X et al (JBC 275, 34818-25, 2000) described inhibition of L- and P-selectin mediated cell adhesion by sulfated saccharides, including carboxyl-reduced and sulfated heparin.

Ikegami-Kuzuhara A. et al. (Brit. J Pharmacol 134, 1498-1504, 2001) described a novel selectin blocker that is a sugar derivative, which inhibited selectin binding to sLex-pentasaccharide ceramide.

Kawashima, H. and Miyasaka, M. (Trends in Glycosciences and Glycotechnology 12, 283-294, 2000) reviewed interactions of CSPGs with selecting. More specifically, ligands for L-selectin were discussed, including binding of versican and heparan sulfate-like molecules.

While the molecules described above demonstrated the utility of selectin blockers for treating inflammation, each has significant drawbacks as a therapeutic agent. These drawbacks include short in vivo half-life, potential immunogenicity, and high cost. A further drawback of these molecules is a lack of efficient means to improve their pharmacological properties.

The present invention discloses methods of screening for small organic compounds capable of inhibiting GAG interaction with GAG-ECAMs. The present invention further discloses that such inhibitor Compounds are useful as inhibitors of cell-matrix and cell-cell adhesion processes and, moreover, are useful for the prevention or treatment of diseases associated with cell adhesion, migration and infiltration.

Using the screening method of the present invention, the anti-L selectin monoclonal antibody DREGG-55 was identified as an inhibitor of the binding of L-selectin to heparin (FIG. 3). DREGG-55 was previously shown to block L-selectin-dependent adhesion in vitro (Co M S et al, 1999 Immunotechnology 493, 253-266). The DREGG-55 antibody is also known to inhibit neutrophil accumulation in vitro and inflammation in vivo. This experiment therefore demonstrates the efficacy of the drug screening method of the present invention for the discovery of compounds capable of inhibiting cell adhesion and having a therapeutic potential.

According to one embodiment, the disorder related to cell adhesion or migration is selected from an inflammatory disorder or process, an autoimmune disorder, cancer, cancer metastasis, and platelet-mediated pathologies.

According to another embodiment, the small organic compounds of the present invention are administered for treating or preventing an inflammatory disorder, condition or process exemplified by, but not restricted to, septic shock, wound associated sepsis, post-ischemic leukocyte-mediated tissue damage (reperfusion injury; such as myocardial or renal ischemia), frost-bite injury or shock, acute leukocyte-mediated lung injury (e.g., adult respiratory distress syndrome), acute pancreatitis, liver cirrhosis, uveitis, asthma, transplantation rejection, graft versus host disease, traumatic shock, stroke, traumatic brain injury, nephritis, acute and chronic inflammation, including atopic dermatitis, psoriasis, and inflammatory bowel disease.

According to another embodiment, the small compounds of the present invention are administered for treating or preventing an autoimmune process or disorder, exemplified by, but not restricted to rheumatoid arthritis, multiple sclerosis, systemic lupus erythematosus, Hashimoto's thyroiditis, Grave's disease, Myasthenia gravis, insulin resistance, and autoimmune thrombocytopenic purpura.

According to a further embodiment, the inhibitor compounds inhibit leukocyte or lymphocyte adhesion, migration or infiltration.

Reperfusion injury is a major problem in clinical cardiology. Therapeutic agents that reduce leukocyte adherence in ischemic myocardium can significantly enhance the therapeutic efficacy of thrombolytic agents. Thrombolytic therapy with agents such as tissue plasminogen activator or streptokinase can relieve coronary artery obstruction in many patients with severe myocardial ischemia prior to irreversible myocardial cell death. However, many such patients still suffer myocardial neurosis despite restoration of blood flow. This “reperfusion injury” is known to be associated with adherence of leukocytes to vascular endothelium in the ischemic zone, presumably in part because of activation of platelets and endothelium by thrombin and cytokines that makes them adhesive for leukocytes (Romson et al., Circulation 67:1016-1023, 1983). These adherent leukocytes can migrate through the endothelium and ischemic myocardium just as it is being rescued by restoration of blood flow.

There are a number of other common clinical disorders in which ischemia and reperfusion results in organ injury mediated by adherence of leukocytes to vascular surfaces including, but not limited to, stroke, mesenteric and peripheral vascular disease, organ transplantation and circulatory shock (in this case many organs might be damaged following restoration of blood flow).

According to one embodiment, platelet-mediated pathologies are exemplified by, but not restricted to atherosclerosis and clotting.

According to another embodiment, the small organic compounds of the present invention are administered for treating or preventing cancer. For certain cancers to spread throughout a patient's body, a process of cell-cell adhesion, or metastasis, must take place. Specifically, cancer cells must migrate from their site of origin and gain access to a blood vessel to facilitate colonization at distant sites. A critical aspect of this process is adhesion of cancer cells (to platelets and to endothelial cells that line the blood vessel wall) a step prior to migrating into surrounding tissue. This process can be interrupted by the administration of inhibitor compounds of the present invention, which, by inhibiting GAG-ECAM interactions, block cell-cell adhesion.

Cancer types that may be treated by the method of the present invention are leukemias that involve extravasation of leukemic cells and tumor formation, such as Acute Myeloid Leukemia. Other types of cancer such as, for example, Hogdkin's lymphoma, non-hodgkin's lymphoma, Burkitt's lymphoma, breast cancer, cervical cancer, ovarian cancer, lung cancer, prostate cancer, colon cancer, and uterine cancer, may be treated by the method of the invention.

According to another embodiment, the small organic compounds of the present invention are administered for treating or preventing angiogenic disorders. The term “angiogenic disorders” as used herein includes conditions involving abnormal neovascularization, such as tumor metastasis and ocular neovascularization including, for example, diabetic retinopathy, neovascular glaucoma, age-related macular degeneration and retinal vein occlusion.

According to yet another embodiment, the small organic compounds of the present invention are administered for treating or preventing of other diseases which involve cell adhesion processes, including, but not limited to, bone degradation, restenosis, eczema, osteoporosis, osteoarthritis and wound healing.

Having now generally described the invention, the same will be more readily understood through reference to the following examples, which are provided by way of illustration and are not intended to limit the scope of the present invention.

### EXAMPLES

### Example 1

### Assay for L-selectin (and P-selectin) Binding to Heparin that is Suitable for Screening of Compound Collections

Porcine intestinal mucosa heparin conjugated to Bovine Serum Albumin (Heparin-BSA; Sigma Cat. No. H0403) at 5 mg/ml in Phosphate Buffered Saline (PBS; pH 6.5) was added to a 96 well polystyrene ELISA plate (NUNC Cat. No. 442404; 0.1 ml per well) and incubated over night (ON) at 4° C. Following the incubation the plate was washed thoroughly by immersion with de-ionized water and PBS (pH 6.5). The ELISA plate was then blocked with BSA (ICN Cat.No. 160069, 3%, 200 μl per well) for 1 hour at room temperature (RT). Following blocking, the plate was washed with de-ionized water and then with PBS (pH 6.5) containing Tween 20 (Sigma Cat. No. P-1379, 0.05% ). Recombinant Human L-Selectin/IgG (Research and Development Systems Cat.No. 728-LS) dissolved in PBS (supplemented with BSA (0.1%) and calcium chloride (1 mM)) was added to the ELISA plate (100 μl per well) and incubated for 60 minutes at RT with shaking. Following incubation, the plate was washed with de-ionized water and three times with PBS (pH 6.5) containing Tween 20. Anti-Human IgG Peroxidase Conjugate (Sigma Product No. A8667) diluted in PBS (supplemented with BSA (0.1%) and calcium chloride (1 mM)), 1:5000, was added to the ELISA plate (100 μl per well) and incubated for 30 minutes at RT with shaking. The plate was then washed with de-ionized water and three times with PBS (pH 6.5) containing Tween. The peroxidase substrate cromogen, TMB (Dako Cat. No. S1599) was added (100 μl per well) to the ELISA plate and incubated at RT. After 15 minutes, ELISA Stop Solution (hydrochloric acid 1N, sulfuric acid 3N) was added (200 μl per well) to stop the peroxidase catalyzed calorimetric reaction. The optical density of the samples was measured at 450 nm using an ELISA plate reader (Dynatech MR5000). FIG. 1 shows a dose response binding of L-selectin to heparin.

The P-selectin assay was carried out in a similar fashion, except that Recombinant Human P-Selectin/IgG (Research and Development Systems Cat.No. 137-PS) was used.

### Example 2

### Inhibition of Human L- and P-Selectin Binding to Immobilized Heparin by Soluble Heparin

Heparin conjugated Bovine Serum Albumin (Hep-BSA; Sigma Cat.No. H0403) at 5 mg/ml in Phosphate Buffered Saline (PBS; Gibco/Invitrogen Cat. No. 1400-067, pH 6.5, 1× concentration)) was added to a 96 well polystyrene ELISA plate (NUNC Cat. No. 442404; 0.1 ml per well) and incubated over night (ON) at 4° C. Following the incubation the plate was washed thoroughly by immersion with de-ionized water and then with PBS (pH 6.5) containing Tween 20 (Sigma Cat. No. P-1379, 0.05%). The Elisa plate was then blocked with BSA (ICN Cat. No. 160069, 3%, 200 μl per well) for 1 hour at Room Temperature (RT). Following blocking, the plate was washed with de-ionized water and then with PBS (pH 6.5) containing Tween 20. Recombinant Human L-Selectin/IgG (Research and Development Systems Cat. No. 728-LS) dissolved in PBS (pH 6.5; supplemented with BSA (0.1%) and CaCl₂ (1 mM)) was incubated with soluble Heparin (Sigma Cat.No. H3393) at different concentrations (0-3000 ng/ml), in separate tubes (final volume 100 μl), each concentration in triplicate, for one hour at RT. Following the incubation, the samples were added to the BSA-blocked Elisa plate and incubated for two hours at RT with shaking. Following incubation, the plate was washed with de-ionized water and three times with PBS (pH 6.5) containing Tween 20. Anti-Human IgG Peroxidase Conjugate (Sigma Product No. A8667) diluted in PBS (supplemented with BSA (0.1%) and CaCl₂ (1 mM)), 1:5000, was added to the Elisa plate (100 μl per well) and incubated for 30 minutes at RT with shaking. The plate was then washed with de-ionized water and three times with PBS (pH 6.5) continuing Tween 20. The peroxidase substrate cromogen, TMB (Dako Cat. No. S1599) was added (100 μl per well) to the Elisa plate and incubated at RT. After 15 minutes, Elisa Stop Solution was added. (200 μl per well) to stop the peroxidase catalyzed colorimetric-reaction. The optical density of the samples was measured at 450 nm using an Elisa plate reader (Dynatech MR5000). FIG. 2 shows a dose dependent inhibition of L-selectin binding to immobilized heparin by soluble heparin. FIG. 4 shows a dose inhibition of P-selectin binding to immobilized heparin by soluble heparin.

### Example 3

### Inhibition of Human L-Selectin Binding to Heparin by Anti-L-Selectin Monoclonal Antibody

Heparin conjugated Bovine Serum Albumin (Hep-BSA; Sigma Cat. No. H0403) at 5 mg/ml in Phosphate Buffered Saline (PBS; Gibco/Invitrogen Cat. No. 1400-067, pH 6.5, 1× concentration) was added to a 96 well polystyrene ELISA plate (NUNC Cat. No. 442404; 0.1 ml per well) and incubated overnight at 4° C. After the incubation, the plate was washed thoroughly by immersion with de-ionized water and then with PBS (pH 6.5) containing Tween 20 (Sigma Cat. No. P-1379, 0.05%). The ELISA plate was then blocked with BSA (ICN Cat. No. 160069, 3%, 200 μl per well) for 1 hour at RT. Following blocking, the plate was washed with de-ionized water and then with PBS (pH 6.5) containing Tween 20. Recombinant Human L-Selectin/IgG dissolved in PBS (pH 6.5; supplemented with BSA (0.1%) and calcium chloride (1 mM)) was incubated separately either with (1) Anti-Human L-Selectin Monoclonal Antibody (DREG-55, Bender MedSystems Cat. No. BMS121; Co M. S. et al., 1999, Immunotechnology 493-4: 253-266); or with (2) Anti-Beta-Amyloid Monoclonal Antibody (Bam 10, Sigma Cat.No. A5213). L-selectin/IgG (10 ng/ml) was incubated with a range of antibody concentrations (5-160 ng/ml) in a final volume of 100 μl ; each concentration in a triplicate for one hour at room temperature. Following the incubation, the samples were added to the BSA-blocked ELISA plate wells and incubated for two hours with shaking. Following incubation, the plate was washed with de-ionized water and three times with PBS (pH 6.5) containing Tween 20. Anti-Human IgG Peroxidase Conjugate (Sigma Product No. A8667) diluted in PBS (supplemented with BSA (0.1%) and calcium chloride (1 mM)), 1:5000, was added to the ELISA plate (100 μl per well) and incubated for 30 minutes at RT with shaking. The plate was then washed with de-ionized water and three times with PBS (pH 6.5) containing Tween 20. The peroxidase substrate cromogen, TMB (Dako Cat. No. S1599) was added (100 μl per well) to the ELISA plate and incubated at room temperature. After 15 minutes, ELISA Stop Solution (hydrochloric acid 1N, sulfuric acid 3N) was added (200 μl per well) to stop the peroxidase catalyzed colorimetric reaction. The optical density of the samples was measured at 450 nm using an ELISA plate reader (Dynatech MR5000). As shown in FIG. 3, anti-human L-selectin antibody exhibited a dose-dependent inhibition of L-selectin binding to immobilized heparin, while anti-beta amyloid antibody was ineffective.

### Example 4

### Assay for GAG-ECAM Protein Binding to HS-GAG that is Suitable for the Screening of Compound Collections

Bovine kidney HS-GAG, shark cartilage chondroitin sulfate, hog skin dermatan sulfate, bovine cornea keratan sulfate and low molecular weight heparins were commercially available (Sigma; Seikagaku Ltd., Japan). Human liver HS-GAG is purified as described (Dudas, J. et al., Biochem. J. 2000, 350, 245-251; Murata K., et al. 1985, Gastroenterology 89, 1248-1257). HS-GAG is conjugated to BSA to prepare a synthetic HS-GAG-BSA complex in which the HS-GAG is coupled via its reducing aldehyde terminus to the protein using sodium cyanoborohydride (Najjam, S. et al. 1997, Cytokine 12, 1013-1022). Other GAGs are coupled to BSA in a similar fashion. The binding assay is similar to the assay described in Example 1. Thus, HS-GAG-BSA is added to a 96 well polystyrene ELISA plate and incubated ON at 4° C. Following the incubation the plate is thoroughly washed and blocked with BSA. Recombinant Human L-Selectin/IgG or any other GAG-ECAM protein such as integrin or interleukin 2 (Najjam, S. et al. 1997, Cytokine 12, 1013-1022), dissolved in PBS (supplemented with BSA (0.1%)) is added to the ELISA plate and incubated for 60 minutes. Following the incubation, the plate is washed, incubated with an antibody specific to L-Selectin/IgG or to any other GAG-ECAM protein such as integrin or interleukin 2, washed again and finally TMB is added to the ELISA plate. After 15 minutes, ELISA Stop Solution is added and the optical density of the samples is measured at 450 nm using an ELISA plate reader.

### Example 5

### A Compound Screening Method—Contacting Test Compounds in the Presence of Heparin (or HS-GAG) and L-Selectin (or GAG-ECAM) to Identify Inhibitor Compounds

The L-selectin (and P-selectin) assay described in Example 1 was used to screen a synthetic chemical compound collection in 96-well plates. A diverse collection of small molecule compounds was purchased from ChemDiv Inc. (San Diego, Calif.). Compounds were dissolved in DMSO at 10 mM and further diluted prior to assay. DMSO concentration in the screening well was up to 2%. Individual compounds at a final concentration of 30 μM were co-incubated with L-selectin/IgG on plates containing immobilized heparin. After washing, bound L-selectin was detected with antibody conjugated to horseradish peroxidase as described in Example 1. Following color development, the % inhibition compared to control (no compound) for every compound was determined. Compounds that inhibited at least 30% of the signal were scored as hits. Dose-response inhibition curves were generated for selected hits and an example is shown in FIG. 5.

Compounds having IC-50) values as indicated in Table 1 are generally suitable for further development and chemical optimization.

TABLE 1
IC-50 Values for Selected Compounds that
Inhibit L-Selectin Binding to Heparin.

| Compound No. | IC-50 [μM] |
|---|---|
| 1 | 15.0 |
| 2 | 2.4 |
| 3 | 3.9 |
| 4 | 5.6 |
| 5 | 13.0 |
| 6 | 1.6 |
| 7 | 7.0 |
| 8 | 2.4 |
| 9 | 1.6 |
| 10 | 0.7 |
| 11 | 13.0 |
| 12 | 5.2 |

A 96-well ELISA assay was also used for measuring P-selectin interaction with heparin for drug screening of collections of compounds. This screening also identified inhibitor compounds with IC-50 values in the 1-10 μM range (Table 2). Example of such inhibitor compound is shown in FIG. 6.

TABLE 2
IC-50 Values for Selected Compounds that
Inhibit P-Selectin Binding to Heparin.

| Compound No. | IC-50 [μM] |
|---|---|
| 21 | 3.2 |
| 22 | 15.2 |
| 23 | 7.6 |
| 24 | 36.0 |
| 25 | 31.0 |
| 26 | 15.0 |
| 27 | 9.0 |
| 28 | 13.0 |
| 29 | 4.0 |

### Example 6

### A Compound Screening Method Using Whole Cell HS-GAG to Identify Inhibitors of the Interaction Between Cellular HS-GAGs and L-Selectin (or Another GAG-ECAM)

Recombinant Human L-Selectin (Research and Development Systems Cat. No. ADP2) is coated onto 96 well polystyrene ELISA plate (NUNC Cat. No. 442404; 0.1 ml per well) and incubated overnight at 4° C. After the incubation the plate is washed thoroughly by immersion with de-ionized water and then with PBS. The ELISA plate is then blocked with BSA (ICN Cat. No. 160069, 3%, 200 μl per well) for 1 hour at RT. Following blocking, the plate is washed with de-ionized water and then with PBS. Individual compounds (10 mM in DMSO) are diluted to a final concentration of 30 μM and added to the plate. Human Umbilical Vein Endothelial Cells (BioWhittaker Cat. No. CC2519; pooled, 3^{rd} passage) are added to the ELISA plate (4×10⁵ cells/ml, 0.1 ml/well) and incubated for 1 hour at 4° C. Following incubation the plate is washed twice with PBS. Anti-Human CD15 Monoclonal Antibody (BRA4F 1, IQ Products, Cat. No. IQP-129P), 1:2000 dilution in PBS containing BSA (0.1%) is added to the ELISA plate and incubated for 30 minutes at 4° C. After incubation the plate is washed twice with PBS and then incubated with Goat Anti-Mouse IgG Peroxidase Conjugated Antibody (Chemicon International, Cat. No. AP124P), 1:5000 dilution in PBS containing BSA (0.1%) for 30 minutes at 4° C. Following incubation, the plate is washed with PBS. The peroxidase substrate cromogen, TMB (Dako Cat. No. S1599) is added (100 μl per well) to the ELISA plate and incubated at RT. After 15 minutes, ELISA Stop Solution is added (200 μl per well) to stop the peroxidase catalyzed colorimetric reaction. The Optical Density of the samples is measured at 450 nm using an ELISA plate reader (Dynatech MR5000).

### Example 7

### A Compound Screening Method Using Whole Cell GAG-ECAM to Identify Inhibitors of the Interaction Between HS-GAGs and GAG-ECAMs

Human neutrophils are fractionated from fresh whole blood according to the published method of Meller et al. (Journal of Clinical Investigation, 1987, 80, 535-544). The viable cell count is made by Trypan Blue exclusion. Heparin-BSA (Sigma Cat. No. H0403) 5 mg/ml in PBS is added to a 96 well polystyrene ELISA plate (NUNC Cat. No. 442404; 0.1 ml per well) and incubated overnight at 4° C. After the incubation the plate is washed thoroughly by immersion with de-ionized water and then with PBS. The ELISA plate is then blocked with BSA (3%, 200 μl per well) for 1 hour at RT. Following blocking, the plate is washed with de-ionized water then twice with PBS. Compounds are dissolved in DMSO at 10 mM concentration, diluted and added to the individual wells at a final concentration of 30 μM. The neutrophil cell suspension (in PBS containing calcium chloride (1 mM), 0.1 ml) is added to the ELISA plate and incubated at 4° C. for 60 minutes. Following the incubation the plate is washed twice with PBS. Anti-Human CD15 Monoclonal Antibody (BRA4F1, IQ Products, Cat. No. IQP-129P), 1:2000 dilution with PBS plus BSA (0.1%) is added to the ELISA plate and incubated for 30 minutes at 4° C. After incubation the plate is washed twice with PBS and then incubated with Goat Anti Mouse IgG Peroxidase Conjugated Antibody (Chemicon International, Cat. No. AP124P, 1:5000 dilution in PBS containing BSA (0.1%)) for 30 minutes at 4° C. After the incubation, the plate is washed with PBS. The peroxidase substrate cromogen, TMB is added (100 μl per well) to the ELISA plate and incubated at RT. After 15 minutes, ELISA Stop Solution is added (200 μl per well) to stop the peroxidase catalyzed colorimetric reaction. The Optical Density of the samples is measured at 450 nm using an ELISA plate reader (Dynatech MR5000).

### Example 8

### Assays to Demonstrate Direct Interaction of Inhibitor Compounds with Heparin and other HS-GAGs

Assay 1.

In order to demonstrate that the inhibitor Compounds bind directly to heparin or to other HS-GAGs, individual compounds were incubated with immobilized heparin in the absence of L-selectin/IgG. 96 well ELISA plates were coated with Heparin-BSA, then blocked with BSA as described in Example 1. Inhibitor Compounds, at a final concentration range of 0.1-200 μM, were incubated in the ELISA plate for 90 min, and then washed with incubation buffer. After washing, L-selectin/IgG was added to these wells. At the same time, in separate control wells, L-selectin was co-incubated with inhibitor Compounds for 90 min and then was added to wells coated with heparin-BSA. Following the incubation, L-selectin bound to the plate was quantified by antibody conjugated to Horse Radish Peroxidase -followed by optical density measurement as described in Example 1. As shown in Table 3, inhibitor Compounds exerted the same level of inhibition on L-selectin binding in the pre-incubation or the co-incubation experiments.

TABLE 3
Direct Binding of Inhibitor Compounds to Heparin

| | Pre-Incubation | Co-Incubation |
|---|---|---|
| Compound no. | % Inhibition | % Inhibition |
| 8 | 70.6 | 77.4 |
| 16 | 68.6 | 75.2 |
| 17 | 58.5 | 61.2 |
| 18 | 40.4 | 44.4 |

Assay 2 .

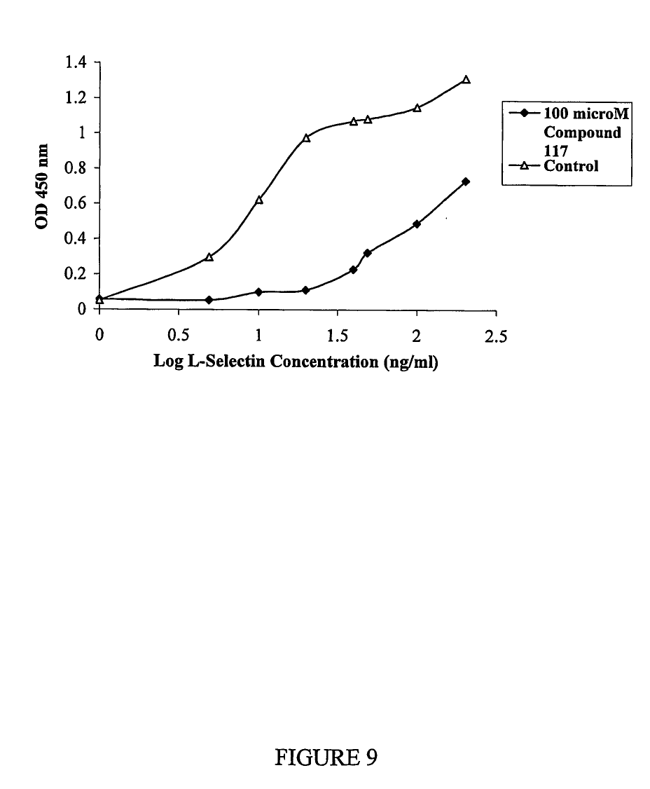

Additional evidence for the direct binding of inhibitor Compounds to heparin and other HS-GAGs was demonstrated by an extension of Assay 1. As in the above assay, 96 well ELISA plates were coated with Heparin-BSA, then blocked with BSA as described in Example 1. L-selectin Hit Compounds, at concentrations predetermined to inhibit L-selectin binding, were incubated in the ELISA plate for 60 min, and then washed with incubation buffer. After washing, L-selectin/IgG was added to the plate pre-incubated with the Hit Compound at increasing concentrations (5-250 μg/ml) and incubated for 90 minutes. Following the incubation, L-selectin bound to the plate was quantified by antibody conjugated to Horse Radish Peroxidase followed by optical density measurement as described in Example 1. Quantitative L-selectin binding was demonstrated at the higher L-selectin concentrations (50-250 μg/ml), demonstrating that the L-selectin inhibitors prevented L-selectin binding to heparin, and therefore these results confirm that the inhibitor Compounds directly interact with heparin, the latter is thus an L-selectin receptor. An example is shown in FIG. 9. The dose response curve delineated with open triangles (Control) represents the results obtained after incubation of the plate with L-selectin in the absence of the inhibitor Compound no. 117.

### Example 9

### A Method for Measurement Leukocyte and Neutrophil Infiltration into Mouse Peritoneum

BALB/c mice (6 weeks old, ˜20 g in weight; 15 mice/group) received intraperitoneal injection of a test compound in 0.2 ml DMSO/Tween/sterile saline 1 hour before administration of thioglycollate (Sigma). Control groups received vehicle, and sham controls received no thioglycollate. Mice were injected intraperitoneally with 1 ml of 3% thioglycollate broth (Xie, X. et al.: J. Biol. Chem., 275, 44, 34818-34825, 2000) Mice were sacrificed after 3 hours, and the peritoneal cavities were lavaged with 5 ml of ice-cold saline containing 2 mM EDTA to prevent clotting. After red blood cell lysis, leukocytes were counted in a hemocytometer. Neutrophils were counted after staining with Türck. Data was expressed as mean±SEM, and statistical analysis was performed by Student t test. A value of P<0.05 was taken to denote statistical significance.

Thioglycollate administration induced approximately 3-fold increase in leukocyte accumulation in the peritoneal cavity. As shown in Table 4, leukocyte migration into the peritoneal cavity was efficiently inhibited by administration of the test compounds at the indicated doses. Similar results were obtained when neutrophil counts were determined.

TABLE 4
Inhibitor Compounds Inhibit Leukocyte (Neutrophil)
Infiltration in Mouse Peritonitis

| Compound no. | Dose (mg/kg) | Leukocytes, % of Control |
|---|---|---|
| 5 | 10 | 56% |
| 7 | 10 | 48% |
| 10 | 10 | 75% |
| 11 | 25 | 32% |
| 21 | 5 | 56% |
| 27 | 1 | 69% |

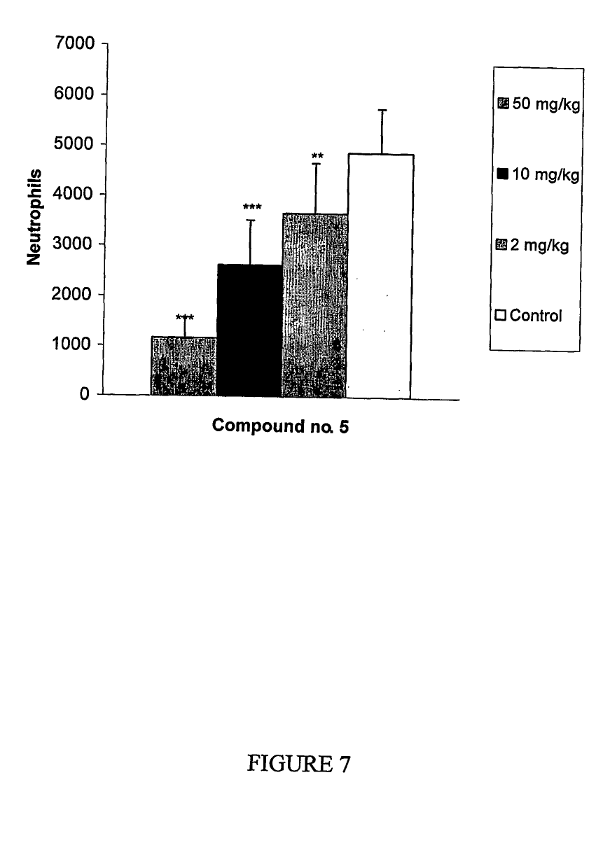

Compound no. 5 was tested in more detail at three doses, 2 mg/kg, 10 mg/kg and 50 mg/kg. A dose response curve for compound no. 5 is shown in FIG. 7. As shown in FIG. 7, compound no. 5 was found to be a potent inhibitor of leukocyte migration; the infiltration was reduced by 75% at a dose of 50 mg/kg, by 50% at 10 mg/kg and by 25% at 2 mg/kg. It should be appreciated that leukocyte migration and infiltration in vivo is a hallmark of inflammatory, autoimmune and other disorders. The ability of these compounds to inhibit leukocyte infiltration in vivo has therefore therapeutic applications for these disorders.

### Example 10

### Delayed-Type Hypersensitivity (DTH)

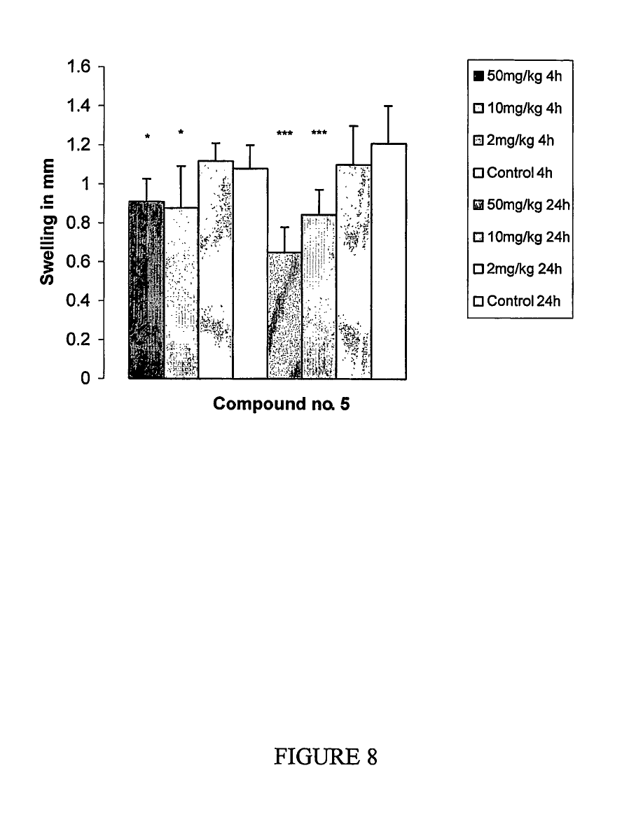

Mice (15 animals per group) were sensitized by topical application of a 2% oxazolone (4-ethoxymethylene-2-phenyl-2-oxazoline-5-one; Sigma, St Louis, Mo.) solution in acetone/olive oil (4:1 vol/vol) to shaved abdomen (50 μl) and to each paw (5 μl) (Lange-Asschenfeldt B. et al., Blood, 2002, 99:538-545). Five days after sensitization, right ears were challenged by topical application of 10 μl of a 1% oxazolone solution, whereas left ears were treated with vehicle alone. Compounds were administered 1 hour prior to challenge. The extent of inflammation was measured 24 hours after challenge, using the mouse ear-swelling test. The unpaired Student t test was used for statistical analyses. FIG. 8 shows that compound no. 5 (at a dose of 3 mg/kg, administered iv) inhibited DTH to 56% of control value 24 hours after challenge. Data were statistically significant at p>0.001.

### Example 11

### A Mouse Model of Kidney Ischemia/Reperfusion

Male Balb/c mice (weighing 20 g; purchased from Velaz, Prague, Czech Republic) are housed individually in standard cages with access to food and water ad libitum (These kinds of studies are approved by the Institutional Animal Care Committee). 30 minutes of unilateral ischemia of the left kidney is followed by contralateral nephrectomy as described in detail previously (Daemen, M. et al., J. Clin. Invest. 104:541-549, 1999). The animals are euthanized at defined time points. At the time of euthanization, blood is collected by orbital puncture, and the left kidney is harvested. Renal neutrophil accumulation is quantified by measuring renal myeloperoxidase content as described. Myeloperoxidase activity is expressed per milligram tissue by comparing the optical density of samples with a horseradish peroxidase titration curve and standardized with respect to wet/dry ratios. Blood urea nitrogen (BUN) content and serum creatinine levels are measured in serum by using a BUN Unimate 5 kit and a CREA MPR3 kit (Boehringer-Mannheim) in a Cobas Fara autoanalyzer (Roche). Kidney specimens are immediately frozen and stored in liquid nitrogen or fixed in buffered formalin and embedded in paraffin. Frozen sections (5 mm) are stained for neutrophils with mAb Gr-1 as described.

### Example 12

### Cecal Ligation and Puncture (CLP)

An animal model for septic shock is described according to Godshall, C. J. et al. (Journal of Surgical Research 102, 45-49, 2002). Male BALB/c mice (20 g) are anesthetized with ketamine (87 mg/g) (Ketaset; Fort Dodge Laboratories, Inc., Fort Dodge, Iowa) and xylazine (13 mg/g) (Rompun; Bayer Corporation, Shawnee Mission, Kans.), and a 2-cm midline incision is made through the linea alba. The cecum is located, ligated with sterile 3-0 silk, and perforated with an 18-gauge needle. A small amount of stool is extruded to ensure wound potency. Sham-treated mice also have surgery done along with cecal manipulations but without ligation and puncture. The cecum is then replaced in its original position within the abdomen, which is closed in two layers. Immediately after surgery, each mouse received a subcutaneous injection of 1 ml of warm normal saline (37° C.) and is placed in an incubator (37° C.) for 15 min. The mice are then moved to a closed room and maintained at 22° C. for the remainder of the experiment. Mice are killed at 4 h after CLP and lung tissue is collected for determination of myeloperoxidase levels.

### Example 13

### Carrageenan-Induced Paw Edema

Acute edema was induced in the left hind paw of Balb/c mice by injecting 0.02 ml of freshly prepared solution of 2% carrageenan after 60 min of test drug administration (Carrageenan-induced paw edema: Torres, S. R. et al., European Journal of Pharmacology 408 2000 199-211). The right paw received 0.02 ml of saline, which served as control. Carrageenan was injected under the plantar region of the right hind paw and the paw thickness was measured at 2, 4 and 24 hours after carrageenan challenge using a Mitutoyo engineer's micrometer expressed as the difference between right and left pad as mean±SEM. As seen in Table 5, test compounds significantly reduced carrageenan induced paw edema after i.p. administration. These results demonstrate that compounds inhibiting GAG binding to GAG-ECAMs display anti-inflammatory activity.

TABLE 5
Examples of Compounds that Inhibited Paw Edema

| Compound no. | Dose (mg/kg) | Paw Edema, % of Control |
|---|---|---|
| 2 | 25 | 66% |
| 5 | 5 | 70% |
| 8 | 5 | 80% |
| 10 | 5 | 66% |
| 11 | 2 | 67% |

### Example 14

### Alternative Assays for GAG-ECAM Binding to GAGs Useful for Compound Screening

Porcine intestinal mucous heparin (Sigma Cat. No. H3393), Bovine Tracheal Chondroitin Sulfate (Calbiochem. Cat. No. 230687), Bovine Kidney heparan sulfate (Sigma Cat. No. H7640) and Porcine Intestinal heparan sulfate (Sigma Cat. No. H9902) conjugated individually to Bovine Serum Albumin (Sigma Cat. No. A7638) were used, as described herein above in Example 1, to demonstrate L-selectin binding to different GAGs. FIG. 10 shows the results of L-selectin binding to the different GAGs and the inhibition of this binding by several different inhibitor Compounds.

### Example 15

### Trinitrobenzine Sulfonic Acid (TNBS) Induced Colitis

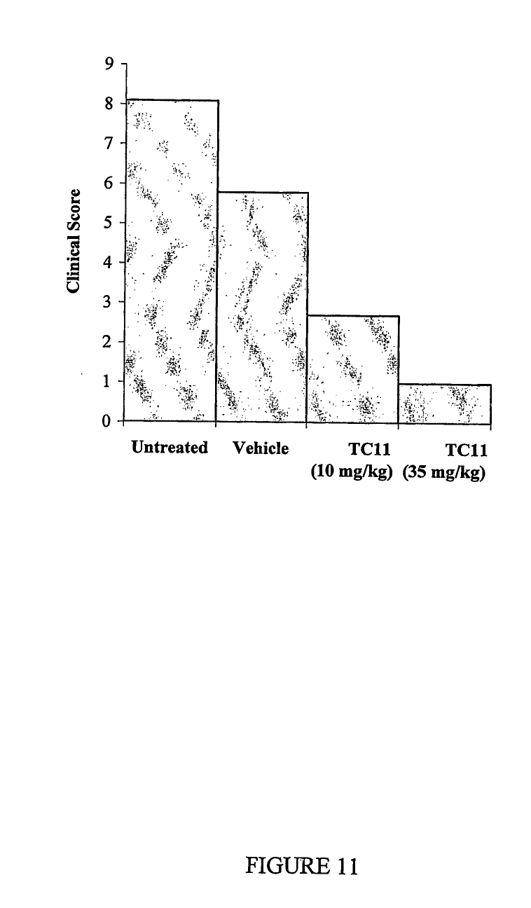

Control mice (12 per group) were injected intraperitoneally (IP) with Test Compound (TC) Vehicle (Tween 80, 5%, 200 μl). Experimental mice (12 per group) were injected IP with TC (10 mg/kg, or 35 mg/kg in 200 μl). The control and experimental mice were injected once per day for 7 successive days. 24 hours after the first IP injection, Inflammatory Bowel Disease (IBD) was induced in the control, experimental, and in an untreated group by intra-rectal administration of TNBS (150 mg/kg dissolved in NaCl (0.9%):EtOH (50%) (1:1; 80 μl mouse). All of the mice were killed by cervical dislocation 7 days after TNBS administration. The colons of the mice were examined under a dissecting microscope (×5) to evaluate the macroscopic lesions on a scale of 0 to 10 (Clinical score). As shown in FIG. 11, inhibitor Compound no. 11 significantly inhibited the colonic damage at doses of 10 and 35 mg/kg.

### Example 16

### Inhibition of Leukocyte Adhesion to Endothelial Cells Under Shear Flow by Inhibitor Compounds

Human T-lymphocytes were passed over a layer of human endothelial cells according to the method of Lawrence and Springer 1991 (Cell 65, pp 859-873). At high shear flow migrating T-lymphocytes adhered transiently and intermittently to the endothelial cells as a consequence of HS-ECAM interaction. The resulting T-cell rolling was recorded by video camera and the number of rolling cells per defined area during a constant period of time was determined. Soluble heparin (competitor of cell surface GAGs) abolished T-cell rolling under high shear flow conditions. Inhibitor Compounds 11, 17 and 18 inhibited T-cell rolling to a level higher than 60%.

It will be appreciated by persons skilled in the art that the present invention is not limited by what has been particularly shown and described herein above. Rather the scope of the invention is defined by the claims that follow.

1. A method of screening for small organic compounds that inhibit the interaction of
GAGs with GAG specific ECAMs, the method comprising the steps of:
a. contacting a GAG with an ECAM in the presence of at least one small organic compound; and
b. measuring the amount of the GAG bound to the ECAM or the amount of the ECAM bound to the GAG, wherein a significant decrease in GAG-ECAM binding in the presence of the compound as compared to GAG-ECAM binding in the absence of said compound identifies the compound as inhibitor compound inhibiting GAG-ECAM interaction.

2. A method of identifying small organic compounds that inhibit the interaction of GAGs with GAG specific ECAMs, the method comprising the steps of:
a. contacting a GAG with at least one small organic compound;
b. removing of unbound organic compound;
c. adding an ECAM; and
d. measuring the amount of the GAG bound to the ECAM or the amount of the ECAM bound to the GAG, wherein a significant decrease in GAG-ECAM binding for the GAG contacted with the organic compound as compared to GAG-ECAM binding for said GAG not contacted with the compound identifies said compound as inhibitor compound inhibiting GAG-ECAM interaction.

3. The method according to claim 1 or 2, wherein the GAG is selected from the group consisting of heparan sulfate (HS-GAG), heparin, chondroitin sulfate, dermatan sulfate, keratan sulfate, derivatives and fragments thereof.

4. The method according to claim 3, wherein the GAG is heparan sulfate (HS-GAG) or heparin.

5. The method according to claim 1 or 2 wherein the GAG is a part of a proteoglycan.

6. The method according to claim 5 wherein the proteoglycan is heparan-sulfate proteoglycan- (HS-PG).

7. The method according to claim 1 or 2 wherein the ECAM is selected from the group consisting of selectins, integrins, fibronectin, and cytokines.

8. The method according to claim 7 wherein the ECAM is L-selectin or P-selectin.

9. The method according to claim 1 or 2 wherein the ECAM is a fusion protein.

10. The method according to claim 1 or 2, wherein the GAG or the ECAM are tagged or labeled.

11. The method according to claim 10 wherein the label is selected from the group consisting of a dye, a fluorescent dye, a chemoluminescent agent, or a radioactive agent.

12. The method according to claim 10 wherein the ECAM is tagged by an antibody.

13. A compound identified according to claim 1 or 2.

14. A pharmaceutical composition comprising as an active ingredient an inhibitor compound capable of inhibiting the interaction of GAGs with GAG specific ECAMs, the compound identified by a screening method comprising the steps of:
a. contacting a GAG with an ECAM in the presence of at least one small organic compound; and
b. measuring the amount of the GAG bound to the ECAM or the amount of the ECAM bound to the GAG, wherein a significant decrease in GAG-ECAM binding in the presence of the compound as compared to GAG-ECAM binding in the absence of said compound identifies the compound as inhibitor compound inhibiting GAG-ECAM interaction,
further comprising a pharmaceutically acceptable diluent or carrier.

15. A pharmaceutical composition comprising as an active ingredient an inhibitor compound capable of inhibiting the interaction of GAGs with GAG specific ECAMs, the compound identified by a screening method comprising the steps of:
a. contacting a GAG with a small organic compound;
b. removing of unbound organic compound;
c. adding an ECAM; and
d. measuring the amount of the GAG bound to the ECAM or the amount of the ECAM bound to the GAG, wherein a significant decrease in GAG-ECAM binding for the GAG contacted with the organic compound as compared to GAG-ECAM binding for said GAG not contacted with the compound identifies said compound as inhibitor compound inhibiting GAG-ECAM interaction,
further comprising a pharmaceutically acceptable diluent or carrier.

16. The pharmaceutical composition according to claim 14 or 15 wherein the GAG is selected from the group consisting of heparan sulfate (HS-GAG), heparin, chondroitin sulfate, dermatan sulfate, keratan sulfate, derivatives and fragments thereof.

17. The pharmaceutical composition according to claim 14 or 15 wherein the ECAM is selected from the group consisting of selecting, integrins, fibronectin, and cytokines.

18. The pharmaceutical composition according to claim 17 wherein the selectin is L-selectin or P-selectin.

19. A method for inhibiting cell adhesion or cell migration comprising the step of exposing a cell to a small organic compound, said compound interacts with at least one GAG in an amount sufficient for preventing the interactions of the GAG with at least one GAG specific ECAM.

20. The method according to claim 19 wherein cell adhesion or cell migration is inhibited in vitro.

21. The method according to claim 19 wherein cell adhesion or cell migration is inhibited in vivo.

22. The method according to claim 19 wherein the GAG is selected from the group consisting of heparan sulfate (HS-GAG), heparin, chondroitin sulfate, dermatan sulfate, keratan sulfate, derivatives and fragments thereof.

23. The method according to claim 22 wherein the GAG is heparan sulfate (HS-GAG) or heparin.

24. The method according to claim 19 wherein the GAG is a part of a proteoglycan.

25. The method according to claim 24 wherein the proteoglycan is heparan-sulfate proteoglycan (HS-PG).

26. The method according to claim 19 wherein the GAG specific ECAM is selected from the group consisting of selecting, integrins, fibronectin, and cytokines.

27. The method according to claim 26 wherein the GAG specific ECAM is P-selectin or L-selectin.

28. A method for modulating anticoagulant activity of glycosaminoglycans in a subject comprising the step of administering a therapeutically effective amount of a pharmaceutical composition according to claim 14 or 15, thereby modulating the anticoagulant activity of glycosaminoglycans.

29. The method according to claim 28, wherein the glycosaminoglycan is heparin.

30. A method for the treatment or prevention of a condition, process, or a disorder related to cell adhesion or migration in a subject comprising the step of administering to a subject in need thereof a pharmaceutical composition comprising as an active ingredient a therapeutically effective amount of a small organic compound, said compound inhibits the interaction of at least one GAG with at least one GAG specific ECAM, thereby preventing cell adhesion or cell migration mediated by the GAG.

31. The method according to claim 30 wherein the process, condition, or disorder related to cell adhesion or migration is selected from the group consisting of inflammatory processes, autoimmune processes, cancer, cancer metastasis, atherosclerosis, and platelet-mediated pathologies.

32. The method according to claim 31 wherein the inflammatory process is selected from the group consisting of septic shock, wound associated sepsis, post-ischemic leukocyte-mediated tissue damage, reperfusion injury, frost-bite injury, shock, acute leukocyte-mediated lung injury, adult respiratory distress syndrome, acute pancreatitis, liver cirrhosis, uveitis, asthma, transplantation rejection, graft versus host disease, traumatic shock, stroke, traumatic brain injury, nephritis, acute and chronic inflammation, atopic dermatitis, psoriasis, and inflammatory bowel disease.

33. The method according to claim 31 wherein the autoimmune process is selected from the group consisting of rheumatoid arthritis, multiple sclerosis, systemic lupus erythematosus, Hashimoto's thyroiditis, Grave's disease, Myasthenia gravis, insulin resistance, and autoimmune thrombocytopenic purpura.

34. The method according to claim 31 wherein the cancer is leukemia.

35. The method according to claim 31 wherein the disease related to cell adhesion or cell migration is selected from the group consisting of bone degradation, restenosis, eczema, osteoporosis, and osteoarthritis and wound healing.
